(19) United States
(12) Patent Application Publication
(10) Pub. No.: US 2012/0238599 A1
(43) Pub. Date: Sep. 20, 2012

(54) HETEROCYCLIC COMPOUNDS AS MEK INHIBITORS

(75) Inventors: Gilnam Lee, Seongnam-si (KR); Han Won Cho, Seoul (KR); Eun Jin Song, Suncheon (KR); Hye Sun Jeon, Seoul (KR); Min Jeong Kim, Sungnam (KR); Jeongbeob Seo, Seongnam-si (KR); Albert Charles Gyorkos, Westminster, CO (US); Suk Young Cho, Longmont, CO (US)

(73) Assignee: CHEMIZON, A DIVISION OF OPTOMAGIC CO., LTD., Seongnam (KR)

(21) Appl. No.: 13/423,898

(22) Filed: Mar. 19, 2012

Related U.S. Application Data
(60) Provisional application No. 61/453,829, filed on Mar. 17, 2011.

(51) Int. Cl. A61K 31/4365; A61K 31/4355; C07D 213/82; A61K 31/4412; A61P 35/00; C07D 498/04; A61K 31/437; C07D 513/04; C12N 9/99; A61P 29/00; C07D 491/048; C07D 495/04

(52) U.S. Cl. 514/301; 546/116; 514/302; 546/291; 514/346; 546/114; 435/184

(57) ABSTRACT

The invention provides novel substituted heterocyclic compounds represented by Formula I and Formula II, or a pharmaceutically acceptable salt, solvate, polymorph, ester, tautomer or prodrug thereof, and a composition comprising these compounds. The compounds provided can be used as inhibitors of MEK and are useful in the treatment of inflammatory diseases, cancer and other hyperproliferative diseases. The invention further provides a method of treatment for inflammatory diseases, cancer and other hyperproliferative diseases in mammals, especially humans.

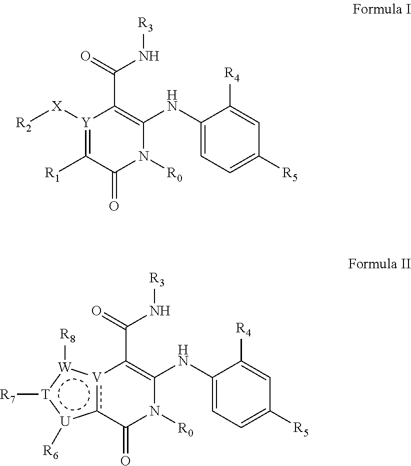

### CROSS-REFERENCE TO RELATED APPLICATION

This application claims the priority to Application No. 61/453,829 filed on Mar. 17, 2011, which is incorporated herein by reference.

### FIELD OF THE INVENTION

This invention relates to a series of substituted heterocyclic compounds which are inhibitors of MEK and are useful in the treatment of inflammatory diseases, cancer and other hyperproliferative diseases. This invention also relates to a pharmaceutical composition comprising the compound of the invention, use of the compound in the preparation of a medicament, and method of treatment for hyperproliferative diseases in mammals, especially humans by administering the compound thereof.

### BACKGROUND OF THE INVENTION

Protein kinases constitute a large family of structurally related enzymes that effect the transfer of a phosphate group from a nucleoside triphosphate to a Ser, Thr or Tyr residue on a protein acceptor. A vast array of cellular functions, including DNA replication, cell cycle progression, energy metabolism, and cell growth and differentiation, are regulated by reversible protein phosphorylation events mediated by protein kinases. Additionally, protein kinase activity has been implicated in a number of diseases, including cancers. Of the >100 dominant oncogenes known to date, many encode receptor and cytoplasmic protein kinases known to be mutated and/or over expressed in human cancers (Blume-Jensen and Hunter, Nature, 411:355-365 (2001)). Accordingly, protein kinase targets have attracted substantial drug discovery efforts in recent years, with several protein kinase inhibitors achieving regulatory approval (reviewed in Fischer, Curr. Med. Chem., 11:1563 (2004); Dancey and Sausville, Nature Rev. Drug Disc., 2:296 (2003)).

The Ras/Raf/MEK/ERK pathway is a central signal transduction pathway, which transmits signals from multiple cell surface receptors to transcription factors in the nucleus which regulate gene expression. This pathway is frequently referred to as the MAP kinase pathway as MAPK stands for mitogen-activated protein kinase indicating that this pathway can be stimulated by mitogens, cytokines and growth factors (Steelman et al., Leukemia 2004, 18, 189-218). Depending upon the stimulus and cell type, this pathway can transmit signals, which result in the prevention or induction of apoptosis or cell cycle progression. The Ras/Raf/MEK/ERK pathway has been shown to play important roles in cell proliferation and the prevention of apoptosis. Aberrant activation of this pathway is commonly observed in malignantly transformed cells Amplification of ras proto-oncogenes and activating mutations that lead to the expression of constitutively active Ras proteins are observed in approximately 30% of all human cancers (Stirewalt et al., Blood 2001, 97, 3589-95). Mutated, oncogenic forms of Ras are found in 50% of colon and >90% pancreatic cancers as well as many other types of cancers (Kohl et al., Science 1993, 260, 1834-1837). The effects of Ras on proliferation and tumorigenesis have been documented in immortal cell lines (McCubrey et al., Int J Oncol 1995, 7, 295-310). bRaf mutations have been identified in more than 60% of malignant melanoma (Davies, H et al., Nature 2002, 417, 949-954). Given the high level of mutations that have been detected at Ras, this pathway has always been considered a key target for therapeutic intervention (Chang et al., Leukemia 2003, 17, 1263-93).

As constitutive or overactivation of MAP kinase cascade plays a pivotal role in cell proliferation and differentiation, inhibition of this pathway is believed is to be beneficial in hyperproliferative diseases. MEK is a key player in this pathway as it is downstream of Ras and Raf. Additionally, it is an attractive therapeutic target because the only known substrates of MEK phosphorylation are the MAP kinases, ERK1 and ERK2 Inhibition of MEK has been shown to have potential therapeutic benefit in several studies. For example, small molecule MEK inhibitors have been shown to inhibit human tumor growth in mouse xenografts, (Seebolt-Leopold et. al., Nature-Medicine, 1999 5(7), 810-816; Trachet et al. AACR Apr. 6-10, 2002, Poster & num; 5426) and inhibit growth of acute myeloid leukemia cells (Milella et. al., J. Clin. Invest., 2001, 108 (6) 851-859).

Compounds suitable as MEK inhibitors are also disclosed in WO 00/41994; WO 00/42022; WO 00/42029; WO 00/68201; WO 01/68619; WO 02/06213, WO 03/077914, WO 05/023251, WO 05/121142, WO07/014011, WO 07/071951, WO 07/123939, WO 08/021389, WO 08/078086, WO 08/120004, WO 08/124085, WO 08/125180, WO 09/018,233, WO07/044084, WO07/121481, WO 09/018238 and WO10108852.

### SUMMARY OF THE INVENTION

This invention provides a compound of formula I or formula II, or a pharmaceutically acceptable salt, solvate, polymorph, ester, tautomer or prodrug thereof:

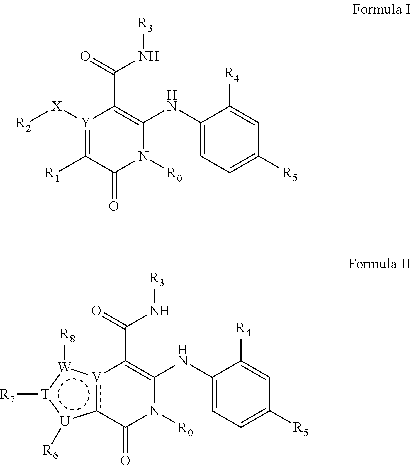

wherein
R₀ is H, C₁-C₆ alkyl, C₃-C₆ cycloalkyl, C₂-C₆ alkenyl, C₅-C₆ cycloalkenyl or C₂-C₆ alkynyl; wherein each alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, and one or two ring carbon atoms of said C₃-C₆ cycloalkyl groups are optionally replaced with, independently, O, N, or S; and
R₁ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl; or
R₁ is —CH₂X′ where X′ represents a group according to formula (III)

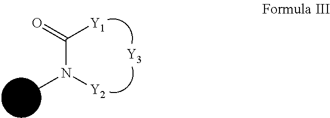

wherein
Y₁ and Y₂ may be the same or different, each represents a single bond, —CO—, —COO, —O—, —OCO—, —NRₐ or —SO₂—;
Y₃ represents a C₁₋₅ alkyl which may be substituted by one to three groups represented by Z;
Z may be the same or different and represent a C₁₋₅ alky group, halogen atom, an oxo group, —ORₐ, —COORₐ, —COOCORₐ, —CO-halogen atom, —OCORₐ, —CONRₐR_{b}, —SRₐ, —SO₂Rₐ, —NRₐR_{b}, —NRₐCOR_{b}, NRₐSO₂R_{b}, —SO₂NRₐR_{b}, a 5 or 6 membered monocyclic or 9 to 13 membered bicyclic heterocyclic group, or a 5 or 6 membered monocyclic or 9 to 13 membered bicyclic heteroaryl group which may be optionally substituted with one or more substituents selected from the group consisting of a C₁₋₅ alkyl group, —ORₐ, and NRₐR_{b}; the alkyl group may be substituted by a hydroxyl group, a C₁₋₅ alkoxy group, or an amino group; the above substituents except the oxo group and the halogen may be linked to each other to form a cycloalkyl group or a heterocyclic group which may has one or more substituents selected from the group consisting of —ORₐ, NRₐR_{b}, and a C₁₋₅ alkyl group that may be substituted with —ORₐ;
Rₐ and R_{b} may be the same or different and each represents a hydrogen atom or a C₁₋₅ alkyl group which may be substituted by one to three groups selected from the group consisting of a hydroxyl group, a C₁₋₅ alkoxy group and an amino group
The symbol “” used in formula III implies the site of bonding; and
X is O, N, S or bond;
R₂ is C₁-C₆ alkyl, C₃-C₆ cycloalkyl, C₂-C₆ alkenyl, C₅-C₆ cycloalkenyl or C₂-C₆ alkynyl; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₂ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl when Y═C or X—R₂=nothing when Y═N; or
R₃ is selected from the group consisting of H, Me, Et, OH, OMe, EtO, HOCH₂CH₂O—, MeCH(OH)CH₂O—, HOCH₂CH(OH)CH₂O—, cyclopropyl-CH₂O—, HOCH₂CH₂O—, HOCH(CH₂CH₃)CH₂O—, HOCH₂C(CH₃)₂CH₂O—, HOCH₂C(CH₃)₂O—, HOCH(CH₃)CH₂O—, MeOCH₂CH₂O—, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heteroarycycloalkyl, heterocyclyl, and heterocyclylalkyl, where each alkyl, alkenyl, alkynyl, cycloalkyl, cycloalkylalkyl, aryl, arylalkyl, heteroaryl, heteroarylalkyl, heterocyclylalkyl, heteroarycycloalkyl, and heterocyclyl is unsubstituted or substituted with 1-3 substituents selected independently from halogen, hydroxyl, C₁-C₄ alkyl, C₁-C₄ alkoxy, cyano, trifluoromethyl, difluoromethoxy, phenyl or substituted phenyl with 1-3 substituents selected independently from halogen, hydroxyl, C₁-C₄ alkyl, C₁-C₄ alkoxy, cyano trifluoromethyl, or difluoromethoxy;
R₄ and R₅ are independently selected from hydrogen, halogen, cyano, nitro, trifluoromethyl, SR₉, OR₉, C(O)R₉, NR₁₀C(O)OR₁₂, OC(O)R₉, NR₁₀S(O)ⱼR₁₂, S(O)ⱼNR₉R₁₀, S(O)ⱼNR₁₀C(O)R₉, C(O)NR₁₀S(O)ⱼR₁₂, S(O)ⱼR₁₂, NR₁₀C(O)R₉, C(O)NR₉R₁₀, NR₁₁C(O)NR₉R₁₀, NR₁₁C(NCN)NR₉R₁₀, NR₉R₁₀ and C₁-C₁₀ alkyl, C₂-C₁₀ alkenyl, C₂-C₁₀ alkynyl, C₃-C₁₀ cycloalkyl, C₃-C₁₀ cycloalkylalkyl, S(O)ⱼ(C₁-C₆ alkyl), S(O)ⱼ(CR₁₀R₁₁)ₘ-aryl, aryl, arylalkyl, heteroaryl, heteroarylalkyl, heterocyclyl, heterocyclylalkyl, O(CR₁₀R₁₀)ₘ-aryl, NR₁₀(CR₁₀R₁₁)ₘ-aryl, O(CR₁₀R₁₁)ₘ-heteroaryl, NR₁₀(CR₁₀R₁₁)ₘ-heteroaryl, O(CR₁₀R₁₁)ₘ-heterocyclyl, NR₁₀(CR₁₀R₁₁)ₘ-heterocyclyl, and S(C₁-C₂ alkyl) optionally substituted with 1-5 fluorine atoms;
R₉ is selected from the group consisting of hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl is unsubstituted or substituted with 1-3 substituents independently selected from the group consisting of halogen, C₁-C₄ alkyl, hydroxyl and amino;
R₁₀ is selected from hydrogen or C₁-C₆ alkyl where alkyl may be unsubstituted or substituted with 1-3 substituents independently selected from the group consisting of halogen, C₁-C₄ alkyl, hydroxyl and amino; or
R₉ and R₁₀ can be taken together with the atom to which they are attached to form a 4 to 10 membered heteroaryl or heterocyclic ring, each of which is unsubstituted or substituted with 1-3 substituents independently selected from the group consisting of halogen, C₁-C₄ alkyl, hydroxyl and amino;
R₁₁ is selected from hydrogen or C₁-C₆ alkyl where alkyl may be unsubstituted or substituted with 1-3 substituents independently selected from the group consisting of halogen, C₁-C₄ alkyl, hydroxyl and amino; or
R₁₀ and R₁₁ can be taken together with the atom to which they are attached to form a 4 to 10 membered carbocyclic, heteroaryl or heterocyclic ring, each of which is unsubstituted or substituted with 1-3 substituents independently selected from the group consisting of halogen, C₁-C₄ alkyl, hydroxyl and amino;
R₁₂ is selected from trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, where each alkyl, cycloalkyl, aryl, heteroaryl and heterocyclyl unsubstituted or substituted with 1-3 substituents independently selected from the group consisting of halogen, C₁-C₄ alkyl, hydroxyl and amino;
m is 0, 1, 2, 3, 4, or 5; and
j is 1 or 2.
T, U, V and W are each independently C, O, N or S to form a heterocycle
R₆ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when U═C and;
R₇ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when V═C and;
R₈ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when W═C and;
n=0, 1, 2, 3 or 4
R_{c}═H, C₁-C₄, C₁-C₆ alkyl, C₃-C₆ cycloalkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; and
R_{d}═H, C₁-C₄, C₁-C₆ alkyl, C₃-C₆ cyclo alkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; or
R_{c} and R_{d} taken together form a 5 or 6 membered heterocyclic group containing 1-2 heteroatoms selected independently from the group consisting of O, N or S and is optionally substituted with 1-2 substituents selectly independently form the group consisting of C₁-C₄ alkyl or C₁-C₄ alkoxy;

In another aspect, the present invention provides some preferable compounds of Formula I or Formula II, wherein R₀ is H or C₁-C₆ alkyl; or a pharmaceutically acceptable salt, solvate, polymorph, ester, tautomer or prodrug thereof.

In another aspect, the present invention provides some preferable compounds of Formula I, wherein R₁ is C₁-C₆ alkyl; or a pharmaceutically acceptable salt, solvate, polymorph, ester, tautomer or prodrug thereof.

In another aspect, the present invention provides some preferable compounds of Formula I, wherein XR₂ is nothing when Y is N; or R₂ is C₁-C₆ alkyl when X is O and Y is C; or a pharmaceutically acceptable salt, solvate, polymorph, ester, tautomer or prodrug thereof.

In another aspect, the present invention provides some preferable compounds of Formula I or Formula II, wherein R₃ is selected from the group consisting of H, Me, Et, OH, OMe, EtO, HOCH₂CH₂O—, MeCH(OH)CH₂O—, HOCH₂CH(OH)CH₂O—, cyclopropyl-CH₂O—, HOCH₂CH₂O—, HOCH(CH₂CH₃)CH₂O—, HOCH₂C(CH₃)₂CH₂O—, HOCH₂C(CH₃)₂O—, HOCH(CH₃)CH₂O—, MeOCH₂CH₂O—, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heteroarycycloalkyl, heterocyclyl, and heterocyclylalkyl, where each alkyl, alkenyl, alkynyl, cycloalkyl, cycloalkylalkyl, aryl, arylalkyl, heteroaryl, heteroarylalkyl, heterocyclylalkyl, heteroarycycloalkyl, and heterocyclyl is unsubstituted or substituted with 1-3 substituents selected independently from halogen, hydroxyl, C₁-C₄ alkyl, C₁-C₄ alkoxy, cyano, trifluoromethyl, difluoromethoxy, phenyl or substituted phenyl with 1-3 substituents selected independently from halogen, hydroxyl, C₁-C₄ alkyl, C₁-C₄ alkoxy, cyano trifluoromethyl, or difluoromethoxy; or a pharmaceutically acceptable salt, solvate, polymorph, ester, tautomer or prodrug thereof.

In another aspect, the present invention provides some preferable compounds of Formula I or Formula II, wherein R₄ and R₅ are independently selected from H or halogen; or a pharmaceutically acceptable salt, solvate, polymorph, ester, tautomer or prodrug thereof.

In another aspect, the present invention provides some preferable compounds of Formula I or Formula II, wherein one of R₄ and R₅ is fluoro, and R₆ is iodo; or a pharmaceutically acceptable salt, solvate, polymorph, ester, tautomer or prodrug thereof.

In another aspect, the present invention provides some more preferable compounds of Formula II, wherein W is O and T, U and V are CR₆ is C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl; and

n=0, 1, 2, 3 or 4
R_{c}═H, C₁-C₄, C₁-C₆ alkyl, C₃-C₆ cyclo alkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; and
R_{d}═H, C₁-C₄, C₁-C₆ alkyl, C₃-C₆ cyclo alkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; or
R_{c} and R_{d} taken together form a 5 or 6 membered heterocyclic group containing 1-2 heteroatoms selected independently from the group consisting of O, N or S and is optionally substituted with 1-2 substituents selectly independently form the group consisting of C₁-C₄ alkyl or C₁-C₄ alkoxy; or a pharmaceutically acceptable salt, solvate, polymorph, ester, tautomer or prodrug thereof.

In other embodiments, the present invention provides compounds represented by the following Formulae:

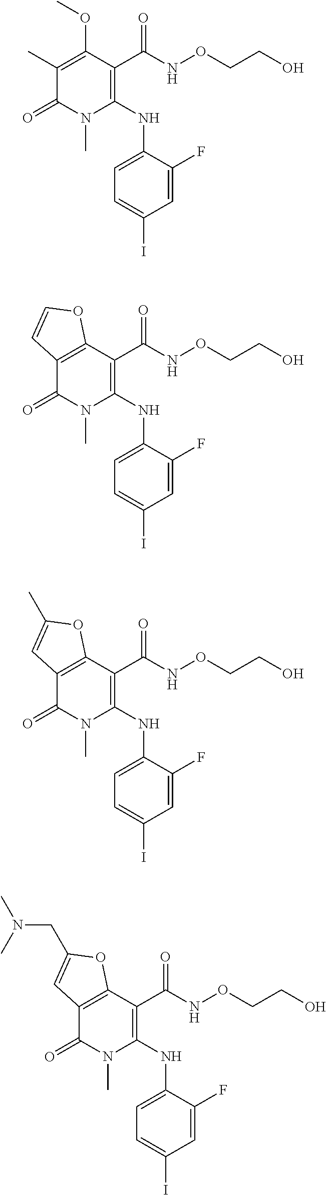

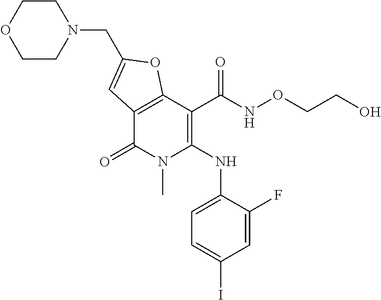

or a pharmaceutically acceptable salt, solvate, polymorph, ester, tautomer or prodrug thereof.

Compounds of present invention are inhibitors of MEK and, consequently, are useful for treating cancers and other hyperproliferative diseases.

In other aspects, the present invention is directed to a pharmaceutical composition comprising an effective amount of a compound of formula I or formula II or a pharmaceutically acceptable salt, solvate, polymorph, ester, tautomer or prodrug thereof. In some embodiments, the pharmaceutical composition further comprises a pharmaceutically acceptable carrier, adjuvants and/or excipients. In some embodiments, such a composition may contain at least one of preservatives, agents for delaying absorption, fillers, binders, adsorbents, buffers, disintegrating agents, solubilizing agents, and other carriers, adjuvants and/or excipients as inert ingredients. The composition may be formulated with a method well-known in the art.

In some aspects, the present invention is directed to a method of treating a disease in an individual suffering from said disease comprising administering to said individual a therapeutically effective amount of a composition comprising a compound of formula I or formula II or a pharmaceutically acceptable salt, solvate, polymorph, ester, tautomer or prodrug thereof.

In other aspects, the present invention is directed to a method of treating a disorder in a mammal, comprising administering to said mammal a therapeutically effective amount of a compound of formula I or formula II or a pharmaceutically acceptable salt, solvate, polymorph, ester, tautomer or pro-drug thereof.

In other aspects, the present invention is directed to a method of treating a disorder in a human, comprising administering to said human a therapeutically effective amount of a compound of formula I or formula II or a pharmaceutically acceptable salt, solvate, polymorph, ester, tautomer or pro-drug thereof.

In other aspects, the present invention is directed to a method of treating an inflammatory disease, condition, or disorder in a mammal, including a human, comprising administering to said mammal a therapeutically effective amount of a compound of formula I or formula II, or a pharmaceutically acceptable salt, ester, prodrug, solvate, such as hydrate, polymorph or tautomer thereof.

In other aspects, the present invention is directed to a method of treating a disorder or condition which is modulated by the MEK cascade in a mammal, including a human, comprising administering to said mammal an amount of the compound of formula I or formula II, or a pharmaceutically acceptable salt, ester, prodrug, solvate, such as hydrate, polymorph or tautomer thereof, effective to modulate said cascade. The appropriate dosage for a particular patient can be determined, according to known methods, by those skilled in the art.

In other aspects, the present invention is directed to use of compound of formula I or formula II or a pharmaceutically acceptable salt, ester, prodrug, solvate, such as hydrate, polymorph or tautomer thereof in the preparation of a pharmaceutical composition. The pharmaceutical composition can be used for treating a disorder or condition which is modulated by the MEK cascade in a mammal, including a human. The pharmaceutical composition is useful for treating cancer, inflammatory disease and other hyperproliferative diseases.

In other aspects, the present invention is directed to a pharmaceutical composition comprising a compound of formula I or formula II or a pharmaceutically acceptable salt, solvate, polymorph, ester, tautomer or prodrug thereof. In some embodiments, the pharmaceutical composition is in a form suitable for oral administration. In further or additional embodiments, the pharmaceutical composition is in the form of a tablet, capsule, pill, powder, sustained release formulation, solution and suspension. In some embodiments, the pharmaceutical composition is in a form suitable for parenteral injection, such as a sterile solution, suspension or emulsion; for topical administration as an ointment or cream or for rectal administration as a suppository. In further or additional embodiments, the pharmaceutical composition is in unit dosage forms suitable for single administration of precise dosages. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.002 to about 6 g/day. In further or additional embodiments the amount of compound of formula I or formula II is about 0.005 to about 5 g/day. In further or additional embodiments the amount of compound of formula I or formula II is about 0.01 to about 5 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the pharmaceutical composition is for administration to a mammal. In further or additional embodiments, the mammal is human. In further or additional embodiments, the pharmaceutical composition further comprises a pharmaceutical carrier, excipient and/or adjuvant. In further or additional embodiments, the pharmaceutical composition further comprises at least one therapeutic agent. In further or additional embodiments, the therapeutic agent is selected from the group consisting of cytotoxic agents, anti-angiogenesis agents and anti-neoplastic agents. In further or additional embodiments, the anti-neoplastic agent is selected from the group consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is taxol, bortezomib or both. In further or additional embodiments, the pharmaceutical composition is administered in combination with an additional therapy. In further or additional embodiments, the additional therapy is radiation therapy, chemotherapy or a combination of both. In further or additional embodiments, the pharmaceutical composition comprises a pharmaceutically acceptable salt of a compound of formula I or formula II.

In other aspects, the present invention is directed to a method for inhibiting a MEK enzyme. The method comprises contacting said MEK enzyme with an amount of a composition comprising a compound of formula I formula II or a pharmaceutically acceptable salt, solvate, polymorph, ester, tautomer or prodrug thereof, sufficient to inhibit said enzyme, wherein said enzyme is inhibited. In some embodiments, the present invention is directed to a method for selectively inhibiting a MEK enzyme.

In other aspects, the present invention is directed to use of a compound of formula I or formula II or a pharmaceutically acceptable salt, solvate, polymorph, ester, tautomer or prodrug thereof in the preparation of a pharmaceutical composition for inhibiting a MEK enzyme.

In further or additional embodiments the enzyme is at least about 1% inhibited. In further or additional embodiments the enzyme is at least about 2% inhibited. In further or additional embodiments the enzyme is at least about 3% inhibited. In further or additional embodiments the enzyme is at least about 4% inhibited. In further or additional embodiments the enzyme is at least about 5% inhibited. In further or additional embodiments the enzyme is at least about 10% inhibited. In further or additional embodiments the enzyme is at least about 20% inhibited. In further or additional embodiments the enzyme is at least about 25% inhibited. In further or additional embodiments the enzyme is at least about 30% inhibited. In further or additional embodiments the enzyme is at least about 40% inhibited. In further or additional embodiments the enzyme is at least about 50% inhibited. In further or additional embodiments the enzyme is at least about 60% inhibited. In further or additional embodiments the enzyme is at least about 70% inhibited. In further or additional embodiments the enzyme is at least about 75% inhibited. In further or additional embodiments the enzyme is at least about 80% inhibited. In further or additional embodiments the enzyme is at least about 90% inhibited. In further or additional embodiments the enzyme is essentially completely inhibited. In further or additional embodiments the MEK enzyme is MEK kinase. In further or additional embodiments the MEK enzyme is MEK1. In further or additional embodiments the MEK enzyme is MEK2. In some embodiments, the compounds of this invention can selectively inhibit a MEK1 enzyme or MEK2 enzyme. In some other embodiments, the compounds of this invention may not have a selectivity between a MEK1 enzyme and MEK2 enzyme. In further or additional embodiments the contacting occurs within a cell. In further or additional embodiments the cell is a mammalian cell. In further or additional embodiments the mammalian cell is a human cell. In further or additional embodiments, the MEK enzyme is inhibited with a composition comprising a pharmaceutically acceptable salt of a compound of formula I or formula II.

In other aspects, the present invention is directed to a method of treatment of a MEK mediated disorder in an individual suffering from said disorder comprising administering to said individual an effective amount of a composition comprising a compound of formula I or formula II or a pharmaceutically acceptable salt, solvate, polymorph, ester, tautomer or prodrug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for treating a MEK mediated disorder.

In some embodiments, the composition comprising a compound of formula I or formula II is administered orally, intraduodenally, parenterally (including intravenous, subcutaneous, intramuscular, intravascular or by infusion), topically or rectally. In some embodiments, the pharmaceutical composition is in a form suitable for oral administration. In further or additional embodiments, the pharmaceutical composition is in the form of a tablet, capsule, pill, powder, sustained release formulations, solution and suspension for oral administration, for parenteral injection as a sterile solution, suspension or emulsion, for topical administration as an ointment or cream, or for rectal administration as a suppository. In further or additional embodiments, the pharmaceutical composition is in unit dosage forms suitable for single administration of precise dosages. In further or additional embodiments, the pharmaceutical composition further comprises a pharmaceutical carrier, excipient and/or adjuvant. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from the MEK mediated disorder is a mammal. In further or additional embodiments, the individual is a human. In some embodiments, the composition comprising a compound of formula I or formula II is administered in combination with an additional therapy. In further or additional embodiments, the additional therapy is radiation therapy, chemotherapy or a combination of both. In further or additional embodiments, the composition comprising a compound of formula I or formula II is administered in combination with at least one therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is selected from taxol, bortezomib or both. In some embodiments, the MEK mediated disorder is selected from the group consisting of inflammatory diseases, infections, autoimmune disorders, stroke, ischemia, cardiac disorder, neurological disorders, fibrogenic disorders, proliferative disorders, hyperproliferative disorders, non-cancer hyper-proliferative disorders, tumors, leukemias, neoplasms, cancers, carcinomas, metabolic diseases, malignant disease, vascular restenosis, psoriasis, atherosclerosis, rheumatoid arthritis, osteoarthritis, heart failure, chronic pain, neuropathic pain, dry eye, closed angle glaucoma and wide angle glaucoma. In further or additional embodiments, the MEK mediated disorder is an inflammatory disease. In further or additional embodiments, the MEK mediated disorder is a hyperproliferative disease. In further or additional embodiments, the MEK mediated disorder is selected from the group consisting of tumors, leukemias, neoplasms, cancers, carcinomas and malignant disease. In further or additional embodiments, the cancer is brain cancer, breast cancer, lung cancer, ovarian cancer, pancreatic cancer, prostate cancer, renal cancer, colorectal cancer or leukemia. In further or additional embodiments, the fibrogenetic disorder is scleroderma, polymyositis, systemic lupus, rheumatoid arthritis, liver cirrhosis, keloid formation, interstitial nephritis or pulmonary fibrosis. In further or additional embodiments, an effective amount of a composition comprising a pharmaceutically acceptable salt of a compound of formula I or formula II is administered.

In other aspects, the present invention is directed to a method for degrading, inhibiting the growth of or killing a cancer cell comprising contacting said cell with an amount of a composition effective to degrade, inhibit the growth of or to kill said cell, the composition comprising a compound of formula I or formula II or a pharmaceutically acceptable salt, solvate, polymorph, ester, tautomer or prodrug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for degrading and/or inhibiting the growth of or killing a cancer cell.

In some embodiments, the cancer cells comprise brain, breast, lung, ovarian, pancreatic, prostate, renal, or colorectal cancer cells. In further or additional embodiments, the composition is administered with at least one therapeutic agent. In further or additional embodiments, the therapeutic agent is taxol, bortezomib or both. In further or additional embodiments, the therapeutic agent is selected from the group consisting of cytotoxic agents, anti-angiogenesis agents and anti-neoplastic agents. In further or additional embodiments, the anti-neoplastic agents selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In some embodiments, the cancer cells are degraded. In further or additional embodiments, 1% of the cancer cells are degraded. In further or additional embodiments, 2% of the cancer cells are degraded. In further or additional embodiments, 3% of the cancer cells are degraded. In further or additional embodiments, 4% of the cancer cells are degraded. In further or additional embodiments, 5% of the cancer cells are degraded. In further or additional embodiments, 10% of the cancer cells are degraded. In further or additional embodiments, 20% of the cancer cells are degraded. In further or additional embodiments, 25% of the cancer cells are degraded. In further or additional embodiments, 30% of the cancer cells are degraded. In further or additional embodiments, 40% of the cancer cells are degraded. In further or additional embodiments, 50% of the cancer cells are degraded. In further or additional embodiments, 60% of the cancer cells are degraded. In further or additional embodiments, 70% of the cancer cells are degraded. In further or additional embodiments, 75% of the cancer cells are degraded. In further or additional embodiments, 80% of the cancer cells are degraded. In further or additional embodiments, 90% of the cancer cells are degraded. In further or additional embodiments, 100% of the cancer cells are degraded. In further or additional embodiments, essentially all of the cancer cells are degraded. In some embodiments, the cancer cells are killed. In further or additional embodiments, 1% of the cancer cells are killed. In further or additional embodiments, 2% of the cancer cells are killed. In further or additional embodiments, 3% of the cancer cells are killed. In further or additional embodiments, 4% of the cancer cells are killed. In further or additional embodiments, 5% of the cancer cells are killed. In further or additional embodiments, 10% of the cancer cells are killed. In further or additional embodiments, 20% of the cancer cells are killed. In further or additional embodiments, 25% of the cancer cells are killed. In further or additional embodiments, 30% of the cancer cells are killed. In further or additional embodiments, 40% of the cancer cells are killed. In further or additional embodiments, 50% of the cancer cells are killed. In further or additional embodiments, 60% of the cancer cells are killed. In further or additional embodiments, 70% of the cancer cells are killed. In further or additional embodiments, 75% of the cancer cells are killed. In further or additional embodiments, 80% of the cancer cells are killed. In further or additional embodiments, 90% of the cancer cells are killed. In further or additional embodiments, 100% of the cancer cells are killed. In further or additional embodiments, essentially all of the cancer cells are killed. In further or additional embodiments, the growth of the cancer cells is inhibited. In further or additional embodiments, the growth of the cancer cells is about 1% inhibited. In further or additional embodiments, the growth of the cancer cells is about 2% inhibited. In further or additional embodiments, the growth of the cancer cells is about 3% inhibited. In further or additional embodiments, the growth of the cancer cells is about 4% inhibited. In further or additional embodiments, the growth of the cancer cells is about 5% inhibited. In further or additional embodiments, the growth of the cancer cells is about 10% inhibited. In further or additional embodiments, the growth of the cancer cells is about 20% inhibited. In further or additional embodiments, the growth of the cancer cells is about 25% inhibited. In further or additional embodiments, the growth of the cancer cells is about 30% inhibited. In further or additional embodiments, the growth of the cancer cells is about 40% inhibited. In further or additional embodiments, the growth of the cancer cells is about 50% inhibited. In further or additional embodiments, the growth of the cancer cells is about 60% inhibited. In further or additional embodiments, the growth of the cancer cells is about 70% inhibited. In further or additional embodiments, the growth of the cancer cells is about 75% inhibited. In further or additional embodiments, the growth of the cancer cells is about 80% inhibited. In further or additional embodiments, the growth of the cancer cells is about 90% inhibited. In further or additional embodiments, the growth of the cancer cells is about 100% inhibited. In further or additional embodiments, a composition comprising a pharmaceutically acceptable salt of a compound of formula I or formula II is used.

In other aspects, the present invention is directed to a method for the treatment or prophylaxis of a proliferative disease in an individual comprising administering to said individual an effective amount of a composition comprising a compound of formula I or formula II or a pharmaceutically acceptable salt, solvate, polymorph, ester, tautomer or pro-drug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for the treatment or prophylaxis of a proliferative disease.

In some embodiments, the proliferative disease is cancer, psoriasis, restenosis, autoimmune disease, or atherosclerosis. In further or additional embodiments, the proliferative disease is a hyperproliferative disease. In further or additional embodiments, the proliferative disease is selected from the group consisting of tumors, leukemias, neoplasms, cancers, carcinomas and malignant disease. In further or additional embodiments, the cancer is brain cancer, breast cancer, lung cancer, ovarian cancer, pancreatic cancer, prostate cancer, renal cancer, colorectal cancer or leukemia. In further or additional embodiments, the fibrogenetic disorder is scleroderma, polymyositis, systemic lupus, rheumatoid arthritis, liver cirrhosis, keloid formation, interstitial nephritis or pulmonary fibrosis. In further or additional embodiments, the cancer is brain cancer, breast cancer, lung cancer, ovarian cancer, pancreatic cancer, prostate cancer, renal cancer, colorectal cancer or leukemia. In further or additional embodiments, the cancer is brain cancer or adrenocortical carcinoma. In further or additional embodiments, the cancer is breast cancer. In further or additional embodiments, the cancer is ovarian cancer. In further or additional embodiments, the cancer is pancreatic cancer. In further or additional embodiments, the cancer is prostate cancer. In further or additional embodiments, the cancer is renal cancer. In further or additional embodiments, the cancer is colorectal cancer. In further or additional embodiments, the cancer is myeloid leukemia. In further or additional embodiments, the cancer is glioblastoma. In further or additional embodiments, the cancer is follicular lymphoma. In further or additional embodiments, the cancer is pre-B acute leukemia. In further or additional embodiments, the cancer is chronic lymphocytic B-leukemia. In further or additional embodiments, the cancer is mesothelioma. In further or additional embodiments, the cancer is small cell line cancer. In some embodiments, the composition comprising a compound of formula I or formula II is administered in combination with an additional therapy. In further or additional embodiments, the additional therapy is radiation therapy, chemotherapy or a combination of both. In further or additional embodiments, the composition comprising a compound of formula I or formula II is administered in combination with at least one therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is selected from taxol, bortezomib or both. In some embodiments, the composition is administered orally, intraduodenally, parenterally (including intravenous, subcutaneous, intramuscular, intravascular or by infusion), topically or rectally. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from the proliferative disease is a mammal. In further or additional embodiments, the individual is a human. In further or additional embodiments, an effective amount of a composition comprising a pharmaceutically acceptable salt of a compound of formula I or formula II is administered.

In other aspects, the present invention is directed to a method for the treatment or prophylaxis of an inflammatory disease in an individual comprising administering to said individual an effective amount of a composition comprising a compound of formula I or formula II or a pharmaceutically acceptable salt, solvate, polymorph, ester, tautomer or prodrug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for the treatment or prophylaxis of an inflammatory disease.

In further or additional embodiments, the inflammatory disease is selected from chronic inflammatory diseases, rheumatoid arthritis, spondyloarthropathies, gouty arthritis, osteoarthritis, juvenile arthritis, acute rheumatic arthritis, enteropathic arthritis, neuropathic arthritis, psoriatic arthritis, pyogenic arthritis, atherosclerosis, systemic lupus erythematosus, inflammatory bowel disease, irritable bowel syndrome, ulcerative colitis, reflux esophagitis, Crohn's disease, gastritis, asthma, allergies, respiratory distress syndrome, pancreatitis, chronic obstructive pulmonary disease, pulmonary fibrosis, psoriasis, eczema or scleroderma. In some embodiments, the composition comprising a compound of formula is administered in combination with an additional therapy. In further or additional embodiments, the composition comprising a compound of formula is administered in combination with at least one therapeutic agent. In some embodiments, the composition is administered orally, intraduodenally, parenterally (including intravenous, subcutaneous, intramuscular, intravascular or by infusion), topically or rectally. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day.

In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from the inflammatory disease is a mammal. In further or additional embodiments, the individual is a human. In further or additional embodiments, an effective amount of a composition comprising a pharmaceutically acceptable salt of a compound of formula I or formula II is administered.

In other aspects, the present invention is directed to a method for the treatment or prophylaxis of cancer in an individual comprising administering to said individual an effective amount of a composition comprising a compound of formula I or formula II or a pharmaceutically acceptable salt, solvate, polymorph, ester, tautomer or prodrug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for the treatment or prophylaxis of a cancer.

In further or additional embodiments, the cancer is brain cancer, breast cancer, lung cancer, ovarian cancer, pancreatic cancer, prostate cancer, renal cancer, colorectal cancer or leukemia. In further or additional embodiments, the fibrogenetic disorder is scleroderma, polymyositis, systemic lupus, rheumatoid arthritis, liver cirrhosis, keloid formation, interstitial nephritis or pulmonary fibrosis. In further or additional embodiments, the cancer is brain cancer, breast cancer, lung cancer, ovarian cancer, pancreatic cancer, prostate cancer, renal cancer, colorectal cancer or leukemia. In further or additional embodiments, the cancer is brain cancer or adrenocortical carcinoma. In further or additional embodiments, the cancer is breast cancer. In further or additional embodiments, the cancer is ovarian cancer. In further or additional embodiments, the cancer is pancreatic cancer. In further or additional embodiments, the cancer is prostate cancer. In further or additional embodiments, the cancer is renal cancer. In further or additional embodiments, the cancer is colorectal cancer. In further or additional embodiments, the cancer is myeloid leukemia. In further or additional embodiments, the cancer is glioblastoma. In further or additional embodiments, the cancer is follicular lymphoma. In further or additional embodiments, the cancer is pre-B acute leukemia. In further or additional embodiments, the cancer is chronic lymphocytic B-leukemia. In further or additional embodiments, the cancer is mesothelioma. In further or additional embodiments, the cancer is small cell line cancer. In some embodiments, the composition comprising a compound of formula I or formula II is administered in combination with an additional therapy. In further or additional embodiments, the additional therapy is radiation therapy, chemotherapy or a combination of both. In further or additional embodiments, the composition comprising a compound of formula I or formula II is administered in combination with at least one therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is selected from taxol, bortezomib or both. In some embodiments, the composition is administered orally, intraduodenally, parenterally (including intravenous, subcutaneous, intramuscular, intravascular or by infusion), topically or rectally. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from cancer is a mammal. In further or additional embodiments, the individual is a human. In further or additional embodiments, an effective amount of a composition comprising a pharmaceutically acceptable salt of a compound of formula I or formula II is administered.

In other aspects, the present invention is directed to a method of reducing the size of a tumor, inhibiting tumor size increase, reducing tumor proliferation or preventing tumor proliferation in an individual, comprising administering to said individual an effective amount of a composition comprising a compound of formula I or formula II or a pharmaceutically acceptable salt, solvate, polymorph, ester, tautomer or prodrug thereof.

In other aspects, the present invention is directed to use of a compound of formula I or formula II or a pharmaceutically acceptable salt, solvate, polymorph, ester, tautomer or prodrug thereof in the preparation of a pharmaceutical composition for reducing the size of a tumor, inhibiting tumor size increase, reducing tumor proliferation or preventing tumor proliferation.

In some embodiments, the size of a tumor is reduced. In further or additional embodiments, the size of a tumor is reduced by at least 1%. In further or additional embodiments, the size of a tumor is reduced by at least 2%. In further or additional embodiments, the size of a tumor is reduced by at least 3%. In further or additional embodiments, the size of a tumor is reduced by at least 4%. In further or additional embodiments, the size of a tumor is reduced by at least 5%. In further or additional embodiments, the size of a tumor is reduced by at least 10%. In further or additional embodiments, the size of a tumor is reduced by at least 20%. In further or additional embodiments, the size of a tumor is reduced by at least 25%. In further or additional embodiments, the size of a tumor is reduced by at least 30%. In further or additional embodiments, the size of a tumor is reduced by at least 40%. In further or additional embodiments, the size of a tumor is reduced by at least 50%. In further or additional embodiments, the size of a tumor is reduced by at least 60%. In further or additional embodiments, the size of a tumor is reduced by at least 70%. In further or additional embodiments, the size of a tumor is reduced by at least 75%. In further or additional embodiments, the size of a tumor is reduced by at least 80%. In further or additional embodiments, the size of a tumor is reduced by at least 85%. In further or additional embodiments, the size of a tumor is reduced by at least 90%. In further or additional embodiments, the size of a tumor is reduced by at least 95%. In further or additional embodiments, the tumor is eradicated. In some embodiments, the size of a tumor does not increase. In some embodiments, tumor proliferation is reduced. In some embodiments, tumor proliferation is reduced by at least 1%. In some embodiments, tumor proliferation is reduced by at least 2%. In some embodiments, tumor proliferation is reduced by at least 3%. In some embodiments, tumor proliferation is reduced by at least 4%. In some embodiments, tumor proliferation is reduced by at least 5%. In some embodiments, tumor proliferation is reduced by at least 10%. In some embodiments, tumor proliferation is reduced by at least 20%. In some embodiments, tumor proliferation is reduced by at least 25%. In some embodiments, tumor proliferation is reduced by at least 30%. In some embodiments, tumor proliferation is reduced by at least 40%. In some embodiments, tumor proliferation is reduced by at least 50%. In some embodiments, tumor proliferation is reduced by at least 60%. In some embodiments, tumor proliferation is reduced by at least 70%. In some embodiments, tumor proliferation is reduced by at least 75%. In some embodiments, tumor proliferation is reduced by at least 80%. In some embodiments, tumor proliferation is reduced by at least 90%. In some embodiments, tumor proliferation is reduced by at least 95%. In some embodiments, tumor proliferation is prevented. In some embodiments, the composition comprising a compound of formula I or formula II is administered in combination with an additional therapy. In further or additional embodiments, the additional therapy is radiation therapy, chemotherapy or a combination of both. In further or additional embodiments, the composition comprising a compound of formula I or formula II is administered in combination with at least one therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is selected from taxol, bortezomib or both. In some embodiments, the composition is administered orally, intraduodenally, parenterally (including intravenous, subcutaneous, intramuscular, intravascular or by infusion), topically or rectally. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from cancer is a mammal. In further or additional embodiments, the individual is a human. In further or additional embodiments, an effective amount of a composition comprising a pharmaceutically acceptable salt of a compound of formula I or formula II is administered.

In other aspects, the present invention is directed to a method for achieving an effect in a patient comprising the administration of an effective amount of a composition comprising a compound of formula I or formula II or a pharmaceutically acceptable salt, solvate, polymorph, ester, tautomer or prodrug thereof, to a patient, wherein the effect is selected from the group consisting of inhibition of various cancers, immunological diseases, and inflammatory diseases. In some embodiments, the effect is inhibition of various cancers. In further or additional embodiments, the effect is inhibition of immunological diseases. In further or additional embodiments, the effect is inhibition inflammatory diseases.

In other aspects, the present invention is directed to use of a compound of formula I or formula II or a pharmaceutically acceptable salt, solvate, polymorph, ester, tautomer or prodrug thereof in the preparation of a pharmaceutical composition for the inhibiting various cancers, immunological diseases, and/or inflammatory diseases.

In some embodiments, the composition comprising a compound of formula I or formula II is administered in combination with an additional therapy. In further or additional embodiments, the additional therapy is radiation therapy, chemotherapy or a combination of both. In further or additional embodiments, the composition comprising a compound of formula I or formula II is administered in combination with at least one therapeutic agent. In some embodiments, the composition is administered orally, intraduodenally, parenterally (including intravenous, subcutaneous, intramuscular, intravascular or by infusion), topically or rectally. In further or additional embodiments the amount of compound of formula I or formula II is in the range of about 0.001 to about 1000 mg/kg body weight/day. In further or additional embodiments the amount of compound of formula I or formula II is in the range of about 0.5 to about 50 mg/kg body weight/day. In further or additional embodiments the amount of compound of formula I or formula II is about 0.001 to about 7 g/day. In further or additional embodiments the amount of compound of formula I or formula II is about 0.01 to about 7 g/day. In further or additional embodiments the amount of compound of formula I or formula II is about 0.02 to about 5 g/day. In further or additional embodiments the amount of compound of formula I or formula II is about 0.05 to about 2.5 g/day. In further or additional embodiments the amount of compound of formula I or formula II is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I or formula II is administered in a single dose, once daily. In further or additional embodiments the compound of formula I or formula II is administered in multiple doses, more than once per day. In further or additional embodiments the compound of formula I or formula II is administered twice daily. In further or additional embodiments the compound of formula I or formula II is administered three times per day. In further or additional embodiments the compound of formula I or formula II is administered four times per day. In further or additional embodiments the compound of formula I or formula II is administered more than four times per day. In some embodiments, the individual suffering from cancer is a mammal. In further or additional embodiments, the individual is a human. In further or additional embodiments, an effective amount of a composition comprising a pharmaceutically acceptable salt of a compound of formula I or formula II is administered.

In other aspects, the present invention is directed to a process for preparing a compound of formula I or formula II or a pharmaceutically acceptable salt, solvate, polymorph, ester, tautomer or prodrug thereof.

### DETAILED DESCRIPTION

The novel features of the invention are set forth with particularity in the appended claims. A better understanding of the features and advantages of the present invention will be obtained by reference to the following detailed description that sets forth illustrative embodiments, in which the principles of the invention are utilized.

While preferred embodiments of the present invention have been shown and described herein such embodiments are provided by way of example only. It should be understood that various alternatives to the embodiments of the invention described herein may be employed in practicing the invention. Those ordinary skilled in the art will appreciate that numerous variations, changes, and substitutions are possible without departing from the invention. It is intended that the following claims define the scope of aspects of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Definition of standard chemistry terms may be found in reference works, including Carey and Sundberg “ADVANCED ORGANIC CHEMISTRY 4 ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, CH₂O is equivalent to OCH₂.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (trans) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. Techniques for inverting or leaving unchanged a particular stereocenter, and those for resolving mixtures of stereoisomers are well known in the art and it is well within the ability of one of skill in the art to choose an appropriate method for a particular situation. See, for example, Fumiss et al. (eds.), VOGEL'S ENCYCLOPEDIA OF PRACTICAL ORGANIC CHEMISTRY 5.sup.TH ED., Longman Scientific and Technical Ltd., Essex, 1991, 809-816; and Heller, Acc. Chem. Res. 1990, 23, 128.

The term “bond” or “single bond” refers to a chemical bond between two atoms, or two moieties when the atoms joined by the bond are considered to be part of larger substructure.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, an optionally substituted group may be un-substituted (e.g., CH₂CH₃), fully substituted (e.g., CF₂CF₃), mono-substituted (e.g., CH₂CH₂F) or substituted at a level anywhere in-between fully substituted and mono-substituted (e.g., CH₂CHF₂, CF₂CH₃, CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons (except in those instances where macromolecular substituents are clearly intended, e.g., polypeptides, polysaccharides, polyethylene glycols, DNA, RNA and the like).

As used herein, C₁-Cn, includes C₁-C₂, C₁-C₃, . . . C₁-Cn. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, isobutyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The terms “heteroatom” or “hetero” as used herein, alone or in combination, refer to an atom other than carbon and hydrogen. Heteroatoms are independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁_{_{—}}₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “alkylene” as used herein, alone or in combination, refers to a diradical derived from the above-defined monoradical, alkyl. Examples include, but are not limited to methylene (—CH₂), ethylene (—CH₂CH₂), propylene (—CH₂CH₂CH₂), isopropylene (—CH(CH₃)CH₂) and the like.

The term “alkenyl” as used herein, alone or in combination, refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (CH═CH₂), 1-propenyl (CH₂CH═CH₂), isopropenyl [C(CH₃)═CH₂], butenyl, 1,3-butadienyl and the like. Whenever it appears herein, a numerical range such as “C₂-C₆ alkenyl” or “C₂_{_{—}}₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkynyl” as used herein, alone or in combination, refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂_{_{—}}₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “heteroalkyl”, “heteroalkenyl” and “heteroalkynyl” as used herein, alone or in combination, refer to optionally substituted alkyl, alkenyl and alkynyl structures respectively, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof.

The terms “halo alkyl”, “halo alkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to an optionally substituted, saturated, hydrocarbon monoradical ring, containing from three to about fifteen ring carbon atoms or from three to about ten ring carbon atoms, though may include additional, non-ring carbon atoms as substituents (e.g. methylcyclopropyl).

A non-limiting example of “cycloalkyl” includes azinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl and quinolizinyl and the like. The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized at-electron system containing 4n+2 n electrons, where n is an integer. Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an optionally substituted aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “heteroaryl” as used herein, alone or in combination, refers to optionally substituted aromatic mono-radicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide and the like.

The term “heterocyclyl” as used herein, alone or in combination, refers collectively to heteroalicyclyl and heteroaryl groups. Herein, whenever the number of carbon atoms in a heterocycle is indicated (e.g., C₁-C₆ heterocycle), at least one non-carbon atom (the heteroatom) must be present in the ring.

Designations such as “C₁-C₆ heterocycle” refer only to the number of carbon atoms in the ring and do not refer to the total number of atoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). For heterocycles having two or more heteroatoms, those two or more heteroatoms can be the same or different from one another. Heterocycles can be optionally substituted. Non-aromatic heterocyclic groups include groups having only three atoms in the ring, while aromatic heterocyclic groups must have at least five atoms in the ring. Bonding (i.e. attachment to a parent molecule or further substitution) to a heterocycle can be via a heteroatom or a carbon atom. The term “alkoxy” as used herein, alone or in combination, refers to an alkyl ether radical, O-alkyl, including the groups O-aliphatic and O-carbocycle, wherein the alkyl, aliphatic and carbocycle groups may be optionally substituted, and wherein the terms alkyl, aliphatic and carbocycle are as defined herein. Non-limiting examples of alkoxy radicals include methoxy, ethoxy, n-propoxy, isopropoxy, n-butoxy, iso-butoxy, sec-butoxy, tertbutoxy and the like.

### Certain Pharmaceutical Terminology

The term “MEK inhibitor” as used herein refers to a compound that exhibits an IC₅₀, with respect to MEK activity, of no more than about 100 μM or not more than about 50 μM, as measured in the Mek1 kinase assay described generally herein. “IC₅₀” is that concentration of inhibitor which reduces the activity of an enzyme (e.g., MEK) to half-maximal level. Compounds described herein have been discovered to exhibit inhibition against MEK. Compounds of the present invention preferably exhibit an IC₅₀ with respect to MEK of no more than about 10 μM, more preferably, no more than about 5 μM, even more preferably not more than about 1 μM, and most preferably, not more than about 200 nM, as measured in the Mek1 kinase assay described herein.

The term “selective,” “selectivity,” or “selectivity” as used herein refers to a compound of this invention having a lower IC₅₀ value for a MEK enzyme as compared to any other enzymes (e.g., at least 2, 5, 10 or more-fold lower). The term may also refer to a compound of this invention having a lower IC₅₀ value for a MEK1 enzyme as compared to a MEK2 enzyme (e.g., at least 2, 5, 10 or more-fold) or alternatively having a lower IC₅₀ value for a MEK2 enzyme as compared to a MEK1 enzyme (e.g., at least 2, 5, 10 or more-fold lower).

The term “subject”, “patient” or “individual” as used herein in reference to individuals suffering from a disorder, a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of a compound into cells or tissues.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, hydroxybutyrate, hydrochloride, hydrobromide, hydro iodide, 2-hydroxyethanesulfonate, iodide, isobutyrate, lactate, maleate, malonate, methanesulfonate, mandelate. metaphosphate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts (See examples at Berge et al., J. Plum. Sci. 1977, 66, 1-19.). Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, IV′ (C₁_{_{—}}₄ alkyl)₄, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra.

The term “solvate” as used herein refers to a combination of a compound of this invention with a solvent molecule formed by solvation. In some situations, the solvate refers to a hydrate, i.e., the solvent molecule is a water molecule, the combination of a compound of this invention and water forms a hydrate.

The term “polymorph” or “polymorphism” as used herein refers to a compound of this invention present in different crystal lattice forms.

The term “ester” as used herein refers to a derivative of a compound of this invention derived from an oxoacid group and a hydroxyl group, either one of which can be present at the compound of this invention.

The term “tautomer” as used herein refers to an isomer readily interconverted from a compound of this invention by e.g., migration of a hydrogen atom or proton.

The term “pharmaceutically acceptable derivative or prodrug” as used herein, refers to any pharmaceutically acceptable salt, ester, salt of an ester or other derivative of a compound of this invention, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Various forms of prodrugs are well known in the art. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. The prodrugs described herein include, but are not limited to, the following groups and combinations of these groups; amine derived prodrugs: Hydroxy prodrugs include, but are not limited to acyloxyalkyl esters, alkoxycarbonyloxyalkyl esters, alkyl esters, aryl esters and disulfide containing esters.

The terms “enhance” or “enhancing,” as used herein, means to increase or prolong either in potency or duration of a desired effect. Thus, in regard to enhancing the effect of therapeutic agents, the term “enhancing” refers to the ability to increase or prolong, either in potency or duration, the effect of other therapeutic agents on a system.

An “enhancing-effective amount,” as used herein, refers to an amount adequate to enhance the effect of another therapeutic agent in a desired system.

The terms “pharmaceutical combination”, “administering an additional therapy”, “administering an additional therapeutic agent” and the like, as used herein, refer to a pharmaceutical therapy resulting from mixing or combining more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

The terms “co-administration”, “administered in combination with” and their grammatical equivalents or the like, as used herein, are meant to encompass administration of the selected therapeutic agents to a single patient, and are intended to include treatment regimens in which the agents are administered by the same or different route of administration or at the same or different times. In some embodiments the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds of the invention and the other agent (s) are administered in a single composition.

The term “metabolite,” as used herein, refers to a derivative of a compound which is formed when the compound is metabolized.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996).

### Synthetic Procedures and Examples

### The Preparation of Compounds of Formula I is Outlined Below:

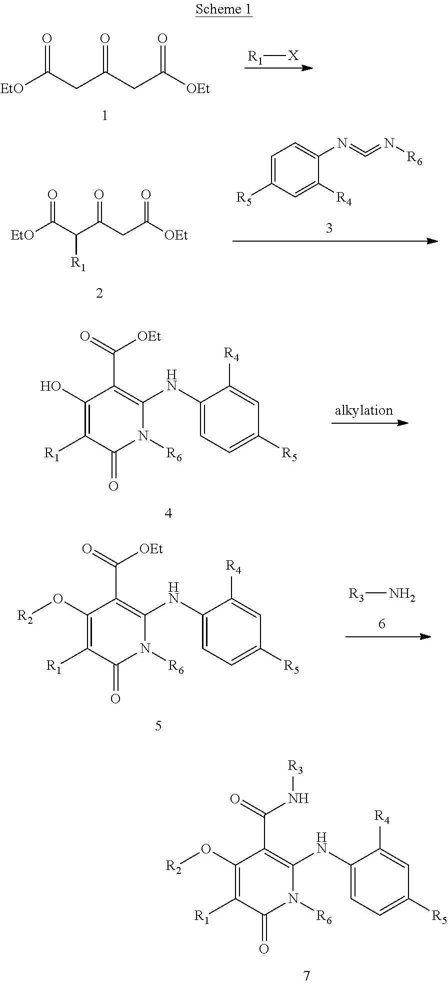

Scheme 1 above illustrates the preparation of pyridone hydroxamate derivatives of (7). Alkylation of diethylacetone 1,3dicarboxylate afford intermediate (2). Condensation with iminoaniline derivatives (3) affords the pyridone (4). The iminoaniline derivatives (3) can be prepared in two steps from anilines by coupling to form the urea followed by reaction with carbon tetrabromide and triphenylphosphine to afford intermediates (3) [Scheme 2].

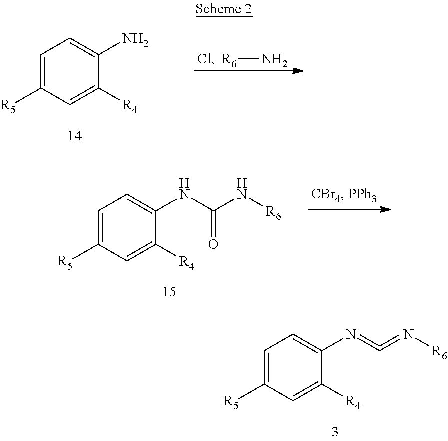

O-alkylation of (4) affords compound (5) which upon treatment with an amine affords the desired compounds (7).

### The Preparation of Compounds of Formula II is Outlined Below:

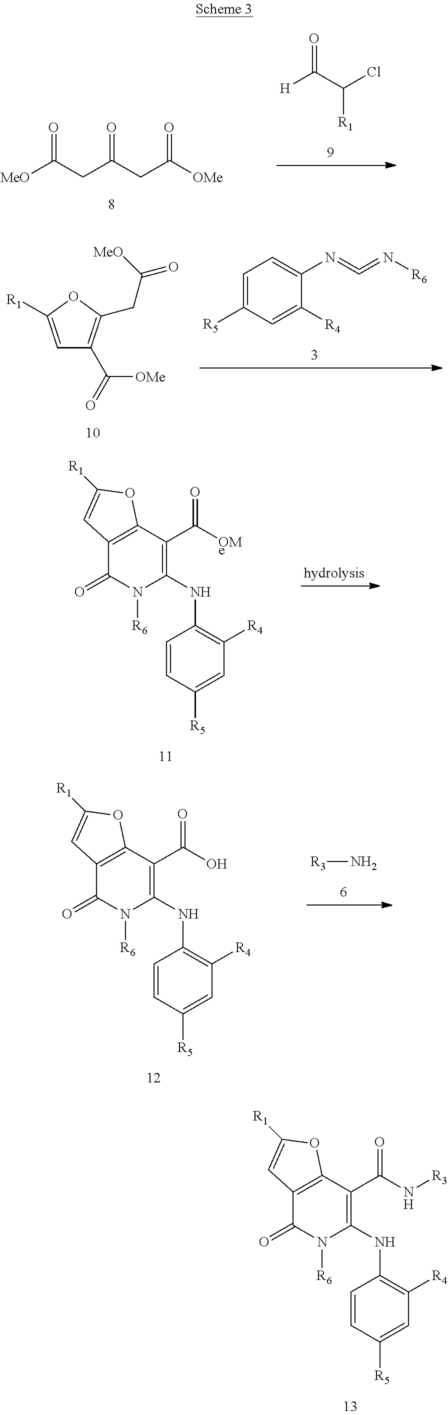

Scheme 3 illustrates the preparation of the dihydrofuro pyridinone derivatives represented by (13). Alkylation of dimethylacetone 1,3dicarboxylate (8) with 2-halo carboxaldehydes (9) affords intermediate (10). Subsequent condensation with iminoaniline derivatives (3) generates the bicyclic dihydro pyridinone (11). Hydrolysis of the ester yields (12) which upon coupling with an amine affords the requisite analogs (13).

### Intermediate 1

### 2-fluoro-4-iodo-N-((methylimino)methylene)aniline

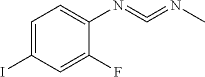

### Step A: 1-(2-fluoro-4-iodophenyl)-3-methylurea

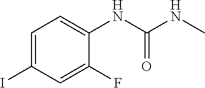

To N,N′-carbonyldiimidazole (51.3 g, 316 mmol) in dry DMF (52 mL) was added TEA (3.55 mL, 25.5 mmol) after addition of a solution of 2-fluoro-4-iodoaniline (50.0 g, 211 mmol) in dry DMF (52 mL) at 0° C. under a N₂ atmosphere. The reaction mixture was stirred at room temperature for 16 h followed by the addition of a solution of 40% methylamine (24.5 g, 316 mmol) at 0° C. After stirring for 1 h at room temperature, the reaction mixture was added to water/toluene (v/v=2/1) while stirring. The resulting solid was collected by filtration, rinsed with water and dried in vacuo to give 1-(2-fluoro-4-iodophenyl)-3-methylurea (57.6 g, 93%) as a white solid, which was used for the next reaction without further purification. ¹H NMR (DMSO-d₆, Varian 400 MHz) δ 2.64 (3H, d, J=2.4 Hz), 6.45-6.49 (1H, m), 7.40-7.42 (1H, m), 7.55 (1H, dd, J=5.4, 2.0 Hz), 7.95 (1H, t, J=8.8 Hz), 8.36 (1H, brs).

### Step B: 2-fluoro-4-iodo-N-((methylimino)methylene)aniline

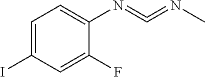

To a solution of 1-(2-fluoro-4-iodophenyl)-3-methylurea (15.0 g, 51.0 mmol) and TEA (28.3 mL, 204 mmol) in DCM (250 mL) was added CBr₄ (33.8 g, 102 mmol) and PPh₃ (26.8 g, 102 mmol) at room temperature. The reaction mixture was stirred at room temperature for 4 h. The solvent was removed by reduce pressure and the residue purified by flash column chromatography on SiO₂ (Hex:EtOAc=20:1 to 5:1) to give 2-fluoro-4-iodo-N-((methylimino)methylene)aniline (9.00 g, 64%) as a red oil. ¹H NMR (CDCl₃, Varian 400 MHz) δ 3.17 (3H, s), 6.78 (1H, t, J=8.4 Hz), 7.33-7.36 (1H, m), 7.38-7.41 (1H, m).

### Intermediate 2

### O-(2-(vinyloxy)ethyl)hydroxylamine

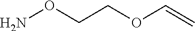

### Step A: 2-(2-(vinyloxy)ethoxy)isoindoline-1,3-dione

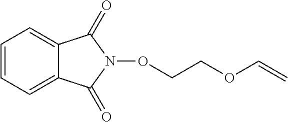

To a solution of 2-(vinyloxy)ethanol (20.4 mL, 227 mmol), triphenylphosphine (59.5 g, 227 mmol), and N-hydroxyphthalimide (37.0 g, 227 mmol) in THF (450 mL) was added DEAD (35.9 mL, 227 mmol) at 0° C. under a N₂ atmosphere. After stirring for 16 h at room temperature, the reaction mixture was concentrated in vacuo. The residue was filtered, washed with chloroform and the filtrate was concentrated in vacuo. The residue was purified by column chromatography on SiO₂ (Hex: EtOAc=2:1) to give 2-(2-(vinyloxy)ethoxy)isoindoline-1,3-dione (32.5 g, 61.4%) as a yellow solid. ¹H NMR (CDCl₃, Varian 400 MHz) δ 4.04-4.08 (3H, m), 4.19 (1H, dd, J=14.4, 2.2 Hz), 4.45-4.48 (2H, m), 6.47 (1H, dd, J=14.0, 6.8 Hz), 7.53-7.78 (2H, m), 7.80-7.87 (2 m, m).

### Step B: O-(2-(vinyloxy)ethyl)hydroxylamine

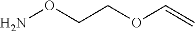

To a solution of 2-(2-(vinyloxy)ethoxy)isoindoline-1,3-dione (32.0 g, 137 mmol) in DCM (96.0 mL) was added dropwise an aqueous solution of methylhydrazine (15.8 mL, 137 mmol) at room temperature. After being stirred for 1 h at room temperature, the resultant suspension was diluted with diethyl ether and filtered. The filtrate was concentrated in vacuo. The residue was purified by column chromatography on SiO₂ (Hex:EtOAc=3:2 to 1:1) to give O-(2-(vinyloxy)ethyl)-hydroxylamine (10.7 g, 76%) as a yellow oil. ¹H NMR (CDCl₃, Varian 400 MHz) δ 3.85-3.93 (4H, m), 4.03 (1H, dd, J=6.8, 2.0 Hz), 4.22 (1H, dd, J=14.2, 2.0 Hz), 5.51 (2H, brs), 6.50 (1H, dd, J=14.2, 6.8 Hz).

### Intermediate 3

### Methyl 6-(2-fluoro-4-iodophenylamino)-5-methyl-4-oxo-4,5-dihydrofuro[3,2-c]pyridine-7-carboxylate

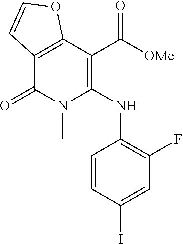

### Step A: methyl 2-(2-methoxy-2-oxoethyl)furan-3-carboxylate

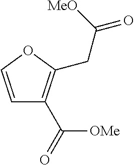

To a solution of dimethyl 3-oxopentanedioate (55.0 g, 316 mmol) in pyridine (113 mL) was added 2-chloropropanal (91.0 g, 524 mmol) dropwise at 0° C. The reaction mixture was stirred at 50° C. for 24 h. The residue was diluted with EtOAc and washed with water and brine, dried over MgSO₄, filtered and concentrated in vacuo. The residue was purified by column chromatography on SiO₂ to give methyl 2-(2-methoxy-2-oxoethyl)furan-3-carboxylate (45.0 g, 72%) as a yellow oil. ¹H-NMR (CDCl₃, Varian, 400 MHz) δ 3.73 (3H, s), 3.83 (3H, s), 4.09 (2H, s), 6.70 (1H, d, J=2.0 Hz), 7.34 (1H, d, J=2.0 Hz).

### Step B: methyl 6-(2-fluoro-4-iodophenylamino)-5-methyl-4-oxo-4,5-dihydrofuro[3,2-c]pyridine-7-carboxylate

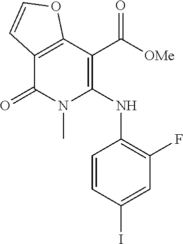

To a solution of methyl 2-(2-methoxy-2-oxoethyl)furan-3-carboxylate (9.94 g, 50.1 mmol) in dry THF (200 mL) was added NaH (55 wt % dispersion in mineral oil, 2.29 g, 52.6 mmol) at 0° C. The reaction mixture was stirred at room temperature for 30 min, and then 2-fluoro-4-iodo-N-((methylimino)methylene)aniline (intermediate 1, 13.8 g, 50.1 mmol) was added slowly with a dropping funnel. The reaction mixture was stirred at room temperature for 3 h. The reaction mixture was quenched with water and extracted with EtoAc and brine (50 mL), dried over Na₂SO₄, filtered and concentrated in vacuo. The residual solid was suspended in water, collected by filtration, rinsed with water and dried in vacuo to give methyl 6-(2-fluoro-4-iodophenylamino)-5-methyl-4-oxo-4,5-dihydrofuro[3,2-c]pyridine-7-carboxylate (11.8 g, 53%) as a yellow solid. ¹H-NMR (CDCl₃, Varian, 400 MHz): δ 3.35 (3H, s), 3.96 (3H, s), 6.45 (1H, t, J=8.4 Hz), 6.96 (2H, d, J=1.2 Hz), 7.36-7.38 (1H, d, J=4.2 Hz), 7.48-7.51 (1H, m), 7.53 (1H, d, J=1.0 Hz), 9.85 (1H, s).

### Intermediate 4

### 2-Chloropropanal

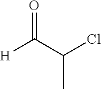

A solution of the 3-chlorobut-1-ene (1.11 mL, 11.0 mmol) in DCM (12.0 mL) was cooled to −60° C. A mixture of O₃/O₂ was then bubbled through the solution for 20 min. The solution was purged with nitrogen, warmed to room temperature, treated with triphenylphosphine (3.48 g, 13.2 mmol) and stirred vigorously for 30 min. The mixture was used for the next reaction without further purification. ¹H-NMR (CDCl₃, Varian, 400 MHz): δ 1.61 (3H, d, J=7.2 Hz), 4.28 (1H, qd, J=1.0, 0.8, 0.8, 0.8 Hz), 9.53 (1H, m).

### Intermediate 5

### Methyl prop-2-ynyl carbonate

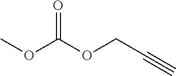

To a cooled (0° C.) and stirred solution of prop-2-yn-1-ol (5.19 mL, 89.0 mmol) and pyridine (14.4 mL, 178 mmol) in diethyl ether (90.0 mL) was added methyl chloroformate (6.91 mL, 89.0 mmol) dropwise over 10 min. The mixture was stirred at room temperature for 15 hours and then dilute hydrochloric acid was added. After extraction with ether, the organic layer was washed with brine and dried over Na₂SO₄, filtered and concentrated in vacuo. The residue was purified by column chromatography on SiO₂ (Hex:EtOAc=3:1) to give methyl prop-2-ynyl carbonate (5.27 g, 52%) as a colorless liquid. ¹H-NMR (CDCl₃, Varian, 400 MHz): δ 2.53 (1H, t, J=2.2 Hz), 3.82 (3H, s), 4.74 (2H, d, J=2.8 Hz).

### Intermediate 6

### 2-Chloro-4-iodo-N-((methylimino)methylene)aniline

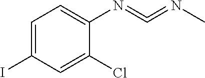

### Step A: 1-(2-chloro-4-iodophenyl)-3-methylurea

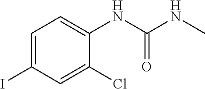

To N,N′-carbonyldiimidazole (480 mg, 2.96 mmol) in dry DMF (0.7 mL) was added TEA (0.33 mL, 2.38 mmol) after addition of a solution of 2-chloro-4-iodoaniline (500 mg, 1.97 mmol) in dry DMF (0.7 mL) at 0° C. under a N₂ atmosphere. The reaction mixture was stirred at room temperature for 16 hour followed by the addition of a solution of 40% methylamine (230 mg, 2.96 mmol) at 0° C. After stiffing for 1 hour at room temperature, the reaction mixture was added to water/toluene (v/v=2/1) while stiffing. The resulting solid was collected by filtration, rinsed with water and dried in vacuo to give 1-(2-chloro-4-iodophenyl)-3-methylurea (530 mg, 87%) as a yellow solid, which was used for the next reaction without further purification. ¹H NMR (DMSO-d₆, Varian 400 MHz) δ 2.64 (3H, d, J=4.8 Hz), 6.90-6.93 (1H, m), 7.54-7.57 (1H, m), 7.73 (1H, d, J=1.6 Hz), 7.97 (1H, d, J=8.8 Hz), 8.07 (1H, s).

### Step B: 2-chloro-4-iodo-N-((methylimino)methylene)aniline

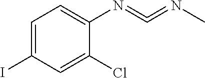

To a solution of 1-(2-chloro-4-iodophenyl)-3-methylurea (530 mg, 51.0 mmol) and TEA (0.95 mL, 6.83 mmol) in DCM (9 mL) was added CBr₄ (1.13 g, 3.41 mmol) and PPh₃ (0.89 g, 3.41 mmol) at room temperature. The reaction mixture was stirred at room temperature for 4 hours. The solvent was removed by reduce pressure and the residue purified by flash column chromatography on SiO₂ (Hex:EtOAc=20:1 to 5:1) to give 2-fluoro-4-iodo-N-((methylimino)methylene)aniline (340 mg, 68%) as a red oil. ¹H NMR (CDCl₃, Varian 400 MHz) δ 3.15 (3H, s), 6.80 (1H, d, J=8.4 Hz), 7.41-7.43 (1H, dd, J=8.2, 2.2 Hz), 7.63-7.68 (1H, m).

### Intermediate 7

### Methyl 6-(2-chloro-4-iodophenylamino)-5-methyl-4-oxo-4,5-dihydrofuro[3,2-c]pyridine-7-carboxylate

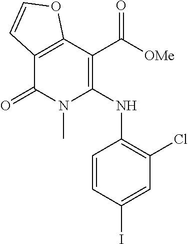

### Step A: methyl 2-(2-methoxy-2-oxoethyl)furan-3-carboxylate

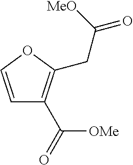

To a solution of dimethyl 3-oxopentanedioate (55.0 g, 316 mmol) in pyridine (113 mL) was added 2-chloropropanal (91.0 g, 524 mmol) dropwise at 0° C. The reaction mixture was stirred at 50° C. for 24 hours. The residue was diluted with EtOAc and washed with water and brine, dried over Na₂SO₄, filtered and concentrated in vacuo. The residue was purified by column chromatography on SiO₂ to give methyl 2-(2-methoxy-2-oxoethyl)furan-3-carboxylate (45.0 g, 72%) as a yellow oil. ¹H-NMR (CDCl₃, Varian, 400 MHz) δ 3.73 (3H, s), 3.83 (3H, s), 4.09 (2H, s), 6.70 (1H, d, J=2.0 Hz), 7.34 (1H, d, J=2.0 Hz).

### Step B: methyl 6-(2-chloro-4-iodophenylamino)-5-methyl-4-oxo-4,5-dihydrofuro[3,2-c]pyridine-7-carboxylate

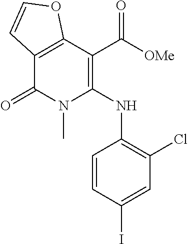

To a solution of methyl 2-(2-methoxy-2-oxoethyl)furan-3-carboxylate (305 mg, 1.53 mmol) in dry THF (8.0 mL) was added NaH (55 wt % dispersion in mineral oil, 67.1 mg, 1.53 mmol) at 0° C. The reaction mixture was stirred at room temperature for 30 min, and then 2-chloro-4-iodo-N-((methylimino)methylene)aniline (intermediate 6, 450 mg, 1.53 mmol) was added slowly. The reaction mixture was stirred at room temperature for 2 hours. The reaction mixture was quenched with water and extracted with EtOAc and brine (50 mL), dried over Na₂SO₄, filtered and concentrated in vacuo. The residual solid was suspended in water, collected by filtration, rinsed with water and dried in vacuo to give methyl 6-(2-chloro-4-iodophenylamino)-5-methyl-4-oxo-4,5-dihydrofuro[3,2-c]pyridine-7-carboxylate (320 mg, 45%) as a yellow solid. ¹H-NMR (DMSO-d₆, Varian, 400 MHz): δ 3.24 (3H, s), 3.76 (3H, s), 6.60 (1H, d, J=8.4 Hz), 7.01-7.02 (1H, m), 7.50-7.52 (1H, dd, J=8.4, 2.0 Hz), 7.87 (1H, m), 7.94 (1H, m), 9.25 (1H, s).

### Intermediate 8

### O-(cyclopropylmethyl)hydroxylamine hydrochloride

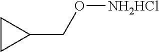

### Step A: 2-(cyclopropylmethoxy)isoindoline-1,3-dione

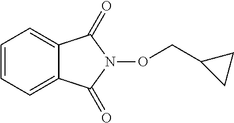

To 2-hydroxyisoindoline-1,3-dione (300 mg, 1.84 mmol) and (bromomethyl)cyclopropane (0.180 mL, 1.84 mmol) in DMF (1.00 mL) was added Et₃N (0.306 mL, 2.21 mmol) dropwise at room temperature. The mixture was stirred at 65° C. for 15 hours. And then the reaction mixture was cooled and precipitate was filtered and washed with water. The solid obtained was dried in vacuo to give 2-(cyclopropylmethoxy)isoindoline-1,3-dione (193 mg, 48%) as a light brown solid. ¹H-NMR (CDCl₃, Varian, 400 MHz): δ 0.38 (2H, m), 0.63 (2H, m), 1.29 (1H, m), 4.05 (2H, d, J=7.2 Hz), 7.75 (2H, m), 7.85 (2H, m).

### Step B: O-(cyclopropylmethyl)hydroxylamine hydrochloride

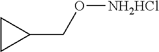

To a solution of 2-(cyclopropylmethoxy)isoindoline-1,3-dione (1.07 g, 4.93 mmol) in DCM (6.00 mL) at room temperature was added N-methylhydrazine sulfate (0.710 g, 4.93 mmol). And the mixture was stirred at room temperature for 1 hour. The reaction mixture was diluted with diethyl ether and filtered. The filtrate was concentrated in vacuo. The residue was suspended in EtOAc and filtered again. 4M HCl in 1,4-Dioxane (1.35 mL, 5.42 mmol) was added to the filtrate and the resulting precipitate was collected by filtration and dried under vacuum to give O-(cyclopropylmethyl)hydroxylamine hydrochloride (55.9 mg, 9.2%) as a yellow solid. ¹H-NMR (DMSO-d₆, Varian, 400 MHz): δ 0.29 (2H, m), 0.57 (2H, m), 1.06 (1H, m), 3.79 (2H, d, J=7.2 Hz), 10.69 (2H, br).

### Intermediate 9

### 2-(Aminooxy)-2-methylpropan-1-ol hydrochloride

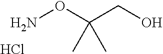

### Step A: ethyl 2-(tert-butoxycarbonylaminooxy)-2-methylpropanoate

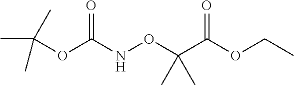

To a solution of tert-butyl hydroxycarbamate (300 mg, 2.25 mmol) in EtOH (17.0 mL) was added KOH (152 mg, 2.70 mmol) and stirred at room temperature till the KOH dissolved into solution. To this was added 2-Bromoisobutyricacid ethyl ester (0.397 mL, 2.70 mmol) and refluxed 15 hours. The white solid was discarded and the filterate was concentrated. The residue was partitioned between water and EtOAc. The combined EtOAc layer was dried with Na₂SO₄, filtered and filtrate was concentrated to give ethyl 2-(tert-butoxycarbonylaminooxy)-2-methylpropanoate (410 mg, 74%) as a colorless oil. ¹H-NMR (CDCl₃, Varian, 400 MHz): δ 1.29 (3H, t, J=1.3 Hz), 1.47 (9H, s), 1.49 (6H, s), 4.20 (2H, q, J=7.1 Hz), 7.37 (1H, s).

### Step B: tert-butyl 1-hydroxy-2-methylpropan-2-yloxycarbamate

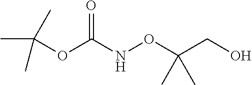

To a solution of ethyl 2-(tert-butoxycarbonylaminooxy)-2-methylpropanoate (410 mg, 1.66 mmol) in anhydrous THF (4.20 mL) at 0° C. under N₂ was added LiAlH₄ (83.0 mg, 2.19 mmol) slowly and stirred for 1 hour. To this was added H₂O (1.00 mL), after aq. NaOH (1.00 mL), H₂O (3.00 mL) and the mixture was stirred for 30 min at room temperature. Then filtered washed with EtOAc, the filtrate was extracted with EtOAc for 3 times, the combined organic extracts were dried, filtered and concentrated in vacuo to give tert-butyl 1-hydroxy-2-methylpropan-2-yloxycarbamate (310 mg, 91%) as a white solid. ¹H-NMR (CDCl₃, Varian, 400 MHz): δ 1.14 (6H, s), 1.42 (9H, s), 3.33 (2H, d, J=7.2 Hz), 4.41 (1H, br). *NH peak was not observed.

### Step C: 2-(aminooxy)-2-methylpropan-1-ol hydrochloride

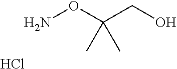

To a solution of tert-butyl 1-hydroxy-2-methylpropan-2-yloxycarbamate (310 mg, 1.51 mmol) in DCM (1.60 mL) was added 4 M HCl in 1,4-Dioxane (2.79 mL, 11.2 mmol) at room temperature and stirred for 1 h. The reaction was concentrated under reduced pressure and the residue was filtered with diethyl ether, and solid was concentrated in vacuum to give 2-(aminooxy)-2-methylpropan-1-ol hydrochloride (236 mg, 110%) as a colorless oil. ¹H-NMR (CDCl₃, Varian, 400 MHz): δ 1.24 (6H, s), 3.48 (2H, s), 10.66 (3H, s). *OH peak was not observed.

### Intermediate 10

### (R)—O-((2,2-dimethyl-1,3-dioxolan-4-yl)methyl)hydroxylamine

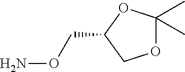

### Step A: (R)-2-((2,2-dimethyl-1,3-dioxolan-4-yl)methoxy)isoindoline-1,3-dione

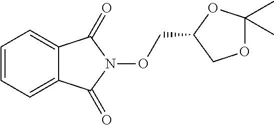

To a solution of (R)-(2,2-dimethyl-1,3-dioxolan-4-yl)methanol (1.00 g, 7.57 mmol), triphenylphosphine (1.99 g, 7.57 mmol), and N-hydroxyphthalimide (1.23 g, 7.57 mmol) in THF (5.2 mL) was added DEAD (2.64 mL, 15.1 mmol) at 0° C. under a N₂ atmosphere. After stiffing for 16 h at room temperature, the reaction mixture was concentrated in vacuo. The residue was filtered, washed with chloroform and the filtrate was concentrated in vacuo. The residue was purified by column chromatography on SiO₂ (Hex: EtOAc=9:1˜1:1) to give (R)-2-((2,2-dimethyl-1,3-dioxolan-4-yl)methoxy)isoindoline-1,3-dione (1.44 g, 68.6%) as a white solid. ¹H NMR (CDCl₃, Varian 400 MHz) δ 1.35 (3H, s), 1.41 (3H, s), 3.98 (1H, dd, J=8.8, 5.2 Hz), 4.12-4.20 (2H, m), 4.32 (1H, dd, J=10.2, 5.8 Hz), 4.47-4.53 (1H, m), 7.74-7.79 (2H, m), 7.83-7.86 (2H, m).

### Step B: (R)—O-((2,2-dimethyl-1,3-dioxolan-4-yl)methyl)hydroxylamine

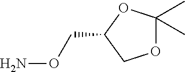

To a solution of (R)-2-((2,2-dimethyl-1,3-dioxolan-4-yl)methoxy)isoindoline-1,3-dione (1.44 g, 5.19 mmol) in DCM (10.4 mL) was added hydrazine hydrate (0.26 g, 5.19 mmol) at room temperature. The reaction mixture was stirred at room temperature for 30 min. The solvent was removed by reduce pressure. The resultant suspension was diluted with diethyl ether and filtered to remove insoluble solid. The filtrate was concentrated in vacuo to give (R)—O-((2,2-dimethyl-1,3-dioxolan-4-yl)methyl)hydroxylamine (700 mg, 92%) as a yellow oil. ¹H NMR (CDCl₃, Varian 400 MHz) δ 1.37 (3H, s), 1.44 (3H, s), 3.68-3.79 (3H, m), 4.05-4.09 (1H, m), 4.32-4.38 (1H, m), 5.56 (2H, brs).

### Example 1

### 6-(2-fluoro-4-iodophenylamino)-N-(2-hydroxyethoxy)-5-methyl-4-oxo-4,5-dihydrofuro[3,2-c]pyridine-7-carboxamide

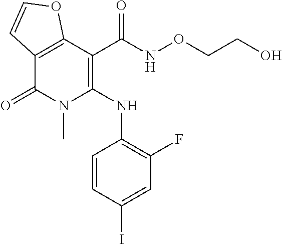

### Step A: 6-(2-fluoro-4-iodophenylamino)-5-methyl-4-oxo-4,5-dihydrofuro[3,2-c]pyridine-7-carboxylic acid

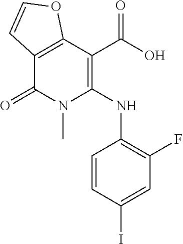

To a solution of methyl 6-(2-fluoro-4-iodophenylamino)-5-methyl-4-oxo-4,5-dihydrofuro[3,2-c]pyridine-7-carboxylate (intermediate 3, 7.00 g, 15.8 mmol) in MeOH (500 mL) was added K₂CO₃ (8.75 g, 63.3 mmol) at room temperature followed by the addition of water (500 mL) via dropping funnel. The reaction mixture was stirred at 70° C. for 3 h. The reaction mixture was quenched with water and then acidified with 10% aq. HCl until pH 1˜2. The resulting solid was collected by filtration, rinsed with water and dried in vacuo to give 6-(2-fluoro-4-iodophenylamino)-5-methyl-4-oxo-4,5-dihydrofuro[3,2-c]pyridine-7-carboxylic acid (5.48 g, 81%) as a yellow solid, which was used for the next reaction without further purification. ¹H-NMR (DMSO-d₆, Varian, 400 MHz) δ 3.25 (3H, s), 6.69 (1H, t, J=8.8 Hz), 6.98 (1H, d, J=1.2 Hz), 7.41 (1H, d, J=4.2 Hz), 7.69 (1H, dd, J=10.6, 1.0 Hz), 7.90 (1H, d, J=1.0 Hz), 9.66 (1H, s), 13.2 (1H, s).

### Step B: 6-(2-fluoro-4-iodophenylamino)-5-methyl-4-oxo-N-(2-(vinyloxy)ethoxy)-4,5-dihydrofuro[3,2-c]pyridine-7-carboxamide

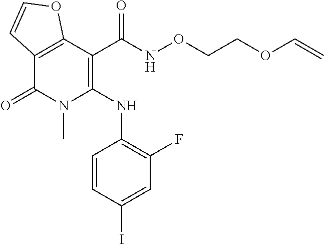

To a solution of 6-(2-fluoro-4-iodophenylamino)-5-methyl-4-oxo-4,5-dihydrofuro[3,2-c]pyridine-7-carboxylic acid (4.00 g, 9.34 mmol) in DMF (110 mL) was added O-(2-(vinyloxy)ethyl)hydroxylamine (intermediate 2, 1.15 g, 11.2 mmol) at room temperature and then was cooled to 0° C. To the reaction mixture was added EDC (2.14 g, 11.2 mmol), HOBT (1.71 g, 11.2 mmol), and TEA (1.56 ml, 11.2 mmol). The mixture was stirred at room temperature for 3 h. The reaction was extracted with EtOAc, washed with water and brine, dried over Na₂SO₄, filtered and concentrated in vacuo to give 6-(2-fluoro-4-iodophenylamino)-5-methyl-4-oxo-N-(2-(vinyloxy)ethoxy)-4,5-dihydrofuro[3,2-c]pyridine-7-carboxamide (2.43 g, 50%) as a white solid. ¹H-NMR (CDCl₃, Varian, 400 MHz): δ 3.32 (3H, s), 3.99-4.01 (2H, m), 4.07-4.27 (2H, m), 4.29-4.31 (2H, m), 6.48-6.55 (2H, m), 7.01 (1H, d, J=1.2 Hz), 7.37 (1H, d, J=4.2 Hz), 7.47 (1H, dd, J=5.0, 1.0 Hz), 7.50 (1H, d, J=1.2 Hz), 10.0 (1H, s), 10.9 (1H, s).

### Step C: 6-(2-fluoro-4-iodophenylamino)-N-(2-hydroxyethoxy)-5-methyl-4-oxo-4,5-dihydrofuro[3,2-c]pyridine-7-carboxamide

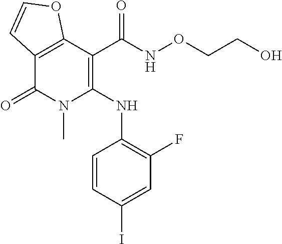

To a solution of 6-(2-fluoro-4-iodophenylamino)-5-methyl-4-oxo-N-(2-(vinyloxy)ethoxy)-4,5-dihydrofuro[3,2-c]pyridine-7-carboxamide (1.57 g, 3.06 mmol) in MeOH (15 mL) was added 2N aq. HCl at room temperature. The mixture was stirred at room temperature for 30 min. The residue was diluted with DCM and washed with water and brine, dried over Na₂SO₄, filtered and concentrated in vacuo to give 6-(2-fluoro-4-iodophenylamino)-N-(2-hydroxyethoxy)-5-methyl-4-oxo-4,5-dihydrofuro[3,2-c]pyridine-7-carboxamide (1.35 g, 91%) as a white solid. ¹H-NMR (CDCl₃, Varian, 400 MHz) δ 3.30 (3H, s), 3.75-3.78 (2H, m), 4.05 (1H, t, J=6.4 Hz), 4.09-4.11 (2H, m), 6.55 (1H, t, J=8.4 Hz), 7.01 (1H, d, J=0.8 Hz), 7.38-7.40 (1H, m), 7.49 (1H, dd, J=4.8, 1.0 Hz), 7.52 (1H, d, J=1.0 Hz), 9.86 (1H, s), 10.8 (1H, s). m/z=487.8 [M+H]⁺

### Example 2

### 2-(2-fluoro-4-iodophenylamino)-N-(2-hydroxyethoxy)-4-methoxy-1,5-dimethyl-6-oxo-1,6-dihydropyridine-3-carboxamide

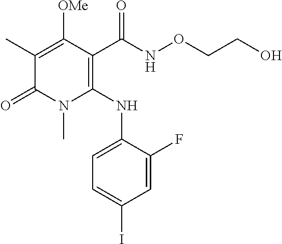

### Step A: diethyl 2-methyl-3-oxopentanedioate

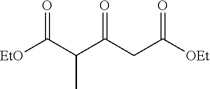

To a solution of diethyl 3-oxopentanedioate (20.0 g, 99.0 mmol) in dry THF (198 mL) was added NaH (55%, 4.53 g, 104 mmol) at 0° C. The mixture was stirred for 30 minutes at 0° C. After dropwise addition of MeI (14.0 g, 99.0 mmol) at 0° C., the reaction mixture was stirred at room temperature for 2 days, and then quenched saturated aq. NH₄Cl (200 mL). The mixture was extracted with EtOAc (3×50 mL). The combined organic layers were washed water (300 mL) and brine (300 mL), dried over Na₂SO₄, filtered and concentrated in vacuo. The residue was purified by column chromatography on SiO₂ (Hex:EtOAc=9:1) to afford diethyl 2-methyl-3-oxopentanedioate (11.1 g, 52%) as a colorless oil. ¹H NMR (CDCl₃, Varian 400 MHz) δ 1.28 (6H, t, J=7.2 Hz), 1.38 (3H, d, J=7.2 Hz), 3.57 (1H, d, J=16.0 Hz), 3.66 (1H, d, J=16.0 Hz), 3.72 (1H, q, J=7.2 Hz), 4.16-4.24 (4H, m).

### Step B: ethyl 2-(2-fluoro-4-iodophenylamino)-4-hydroxy-1,5-dimethyl-6-oxo-1,6-dihydropyridine-3-carboxylate

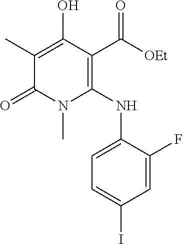

To a solution of diethyl 2-methyl-3-oxopentanedioate (9.56 g, 44.2 mmol) in dry THF (88 mL) was added NaH (55%, 2.02 g, 46.4 mmol) at 0° C. The mixture was stirred for 30 minutes at 0° C. After dropwise addition of 2-fluoro-4-iodo-N-((methylimino)methylene)aniline (intermediate 1, 12.2 g, 44.2 mmol) at 0° C., the reaction mixture was stirred at room temperature overnight, and then quenched with saturated 1N aq. HCl (60 mL). The mixture was extracted with EtOAc (3×30 mL). The combined organic layers were washed water (100 mL) and brine (100 mL), dried over Na₂SO₄, filtered and concentrated in vacuo. The residue was purified by column chromatography on SiO₂ (Hex:EtOAc=7:3 to 1:1) to afford ethyl 2-(2-fluoro-4-iodophenylamino)-4-hydroxy-1,5-dimethyl-6-oxo-1,6-dihydropyridine-3-carboxylate (2.98 g, 15%) as a yellow oil. ¹H NMR (CDCl₃, Varian 400 MHz) δ 1.36 (3H, t, J=7.2 Hz), 2.05 (3H, s), 3.35 (3H, s), 4.40 (2H, q, J=7.2 Hz), 6.33 (1H, t, J=8.4 Hz), 7.36 (1H, d, J=8.4 Hz), 7.50 (1H, dd, J=10.0, 2.0 Hz), 8.28 (1H, brs), 11.20 (1 m, brs).

### Step C: ethyl 2-(2-fluoro-4-iodophenylamino)-4-methoxy-1,5-dimethyl-6-oxo-1,6-dihydropyridine-3-carboxylate

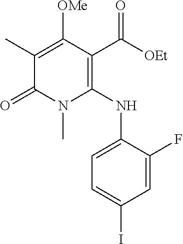

A mixture of ethyl 2-(2-fluoro-4-iodophenylamino)-4-hydroxy-1,5-dimethyl-6-oxo-1,6-dihydropyridine-3-carboxylate (2.78 g, 6.23 mmol), dimethyl sulfate (0.60 mL, 6.23 mmol) and K₂CO₃ (1.72, 12.5 mmol) in acetone (31 mL) was refluxed for 1 hour, cooled to room temperature, and then partitioned between EtOAc and water. The separated aqueous layer was extracted with EtOAc. The combined organic layers were washed with brine, dried over Na₂SO₄, filtered and concentrated in vacuo. The residue was purified by column chromatography on SiO₂ (Hex:EtOAc=3:2) to afford ethyl 2-(2-fluoro-4-iodophenylamino)-4-methoxy-1,5-dimethyl-6-oxo-1,6-dihydropyridine-3-carboxylate (1.04 g, 36%) as a yellow oil. ¹H NMR (CDCl₃, Varian 400 MHz) δ 1.35 (3H, t, J=7.2 Hz), 2.09 (3H, s), 3.32 (3H, s), 3.78 (3H, s), 4.31 (2H, q, J=7.2 Hz), 6.35 (1H, t, J=8.4 Hz), 7.34 (1H, d, J=8.4 Hz), 7.46 (1H, dd, J=10.0, 2.0 Hz), 8.88 (1H, brs).

### Step D: 2-(2-fluoro-4-iodophenylamino)-4-methoxy-1,5-dimethyl-6-oxo-N-(2-(vinyloxy)ethoxy)-1,6-dihydropyridine-3-carboxamide

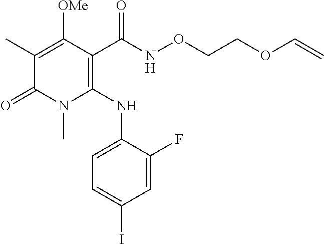

To a mixture of ethyl 2-(2-fluoro-4-iodophenylamino)-4-methoxy-1,5-dimethyl-6-oxo-1,6-dihydropyridine-3-carboxylate (1.29 g, 2.80 mmol) and O-(2-(vinyloxy)ethyl)hydroxylamine (intermediate 2, 433 mg, 4.20 mmol) in dry THF (14 mL) was added LiHMDS (16.8 mL, 16.8 mmol, 1.0 M solution in hexane) at 0° C. The reaction mixture was stirred for 2 hours at room temperature, and then quenched with saturated 1N aq. HCl (50 mL). The mixture was extracted with EtOAc (3×20 mL). The combined organic layers were washed with water (50 mL) and brine (50 mL), dried over Na₂SO₄, filtered and concentrated in vacuo. The residue was purified by column chromatography on SiO₂ (Hex:EtOAc=1:1 to 1:2) to afford 2-(2-fluoro-4-iodophenylamino)-4-methoxy-1,5-dimethyl-6-oxo-N-(2-(vinyloxy)ethoxy)-1,6-dihydropyridine-3-carboxamide (850 mg, 54%) as a yellow solid. ¹H NMR (CDCl₃, Varian 400 MHz) δ 2.08 (3H, s), 3.27 (3H, s), 3.79 (3H, s), 3.94-3.96 (2H, m), 4.06 (1H, dd, J=6.8, 2.4 Hz), 4.19-4.26 (3H, m), 6.41 (1H, t, J=8.4 Hz), 6.50 (1H, dd, J=14.6, 6.8 Hz), 7.34 (1H, d, J=8.4 Hz), 7.45 (1H, dd, J=10.0, 2.0 Hz), 10.18 (1H, brs), 10.52 (1H, brs). m/z=517.9 [M+H]⁺

### Step E: 2-(2-fluoro-4-iodophenylamino)-N-(2-hydroxyethoxy)-4-methoxy-1,5-dimethyl-6-oxo-1,6-dihydropyridine-3-carboxamide

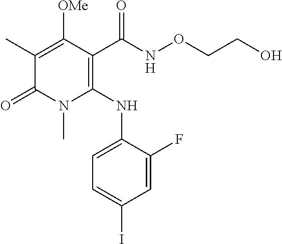

To a solution of 2-(2-fluoro-4-iodophenylamino)-4-methoxy-1,5-dimethyl-6-oxo-N-(2-(vinyloxy)ethoxy)-1,6-dihydropyridine-3-carboxamide (400 mg, 0.773 mmol) in MeOH (8.0 mL) was added 2M aq. HCl (2.3 mL, 4.64 mmol) at room temperature. The reaction mixture was stirred for 15 minutes at room temperature, and then concentrated in vacuo. The residue was dissolved in DCM, neutralized with saturated aq. NaHCO₃ at 0° C. The separated aqueous layer was extracted with DCM. The combined organic layers were washed with brine, dried over Na₂SO₄, filtered and concentrated in vacuo. The residual solid was suspended in Et₂O, collected by filtration, and washed with Et₂O to afford 2-(2-fluoro-4-iodophenylamino)-N-(2-hydroxyethoxy)-4-methoxy-1,5-dimethyl-6-oxo-1,6-dihydropyridine-3-carboxamide (357 mg, 94%) as a yellow solid. ¹H NMR (DMSO-d₆, Varian 400 MHz) δ 1.95 (3H, s), 3.25 (3H, s), 3.40-3.46 (2H, m), 3.59-3.61 (2H, m), 3.73 (3H, s), 4.64 (1H, brs), 6.40 (1H, t, J=8.8 Hz), 7.31 (1H, dd, J=8.4, 1.2 Hz), 7.45 (1H, dd, J=10.0, 2.0 Hz), 8.25 (1H, brs), 11.13 (1H, brs). m/z=491.9 [M+H]⁺

### Example 3

### 6-(2-fluoro-4-iodophenylamino)-N-(2-hydroxyethoxy)-2,5-dimethyl-4-oxo-4,5-dihydrofuro[3,2-c]pyridine-7-carboxamide

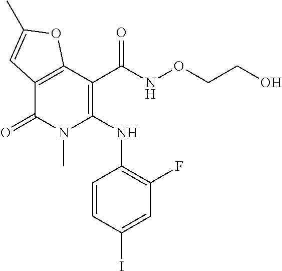

### Step A: methyl 5-ethyl-2-(2-methoxy-2-oxoethyl)furan-3-carboxylate

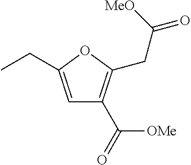

To a solution of dimethyl 3-oxopentanedioate (1.30 mL, 8.83 mmol) in pyridine (2.28 mL) was added crude 2-chloropropanal (intermediate 4, 817 mg, 8.83 mmol) dropwise at 0° C. The reaction mixture was stirred at 40° C. for 15 hours. The residue was diluted with DCM and washed with water and brine, dried over MgSO₄, filtered and concentrated in vacuo. The residue was purified by column chromatography on SiO₂ (Hex:EtOAc=5:1) to give methyl 5-ethyl-2-(2-methoxy-2-oxoethyl)furan-3-carboxylate (420 mg, 22%) as a colorless liquid. ¹H-NMR (CDCl₃, Varian, 400 MHz): δ 2.28 (3H, s), 3.72 (3H, s), 3.80 (3H, s), 4.02 (2H, s), 6.27 (1H, s).

### Step B: methyl 6-(2-fluoro-4-iodophenylamino)-2,5-dimethyl-4-oxo-4,5-dihydrofuro[3,2-c]pyridine-7-carboxylate

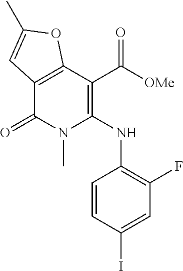

To a solution of 2-fluoro-4-iodo-N-((methylimino)methylene)aniline (intermediate 1, 1.22 g, 5.75 mmol) in dry THF (25.0 mL) was added NaH (55 wt % dispersion in mineral oil, 0.263 g, 6.04 mmol) at 0° C. The reaction mixture was stirred at room temperature for 30 min, and then methyl 5-ethyl-2-(2-methoxy-2-oxoethyl)furan-3-carboxylate (1.59 g, 5.75 mmol) was added slowly with dropping funnel. The reaction mixture was stirred at room temperature for 15 hours. The reaction mixture was quenched with water, extracted with EtOAc. The resulting solid was collected by filtration, rinsed with water and dried in vacuo to give methyl 6-(2-fluoro-4-iodophenylamino)-2,5-dimethyl-4-oxo-4,5-dihydrofuro[3,2-c]pyridine-7-carboxylate (833 mg, 32%) as a yellow solid, which was used for the next reaction without further purification. ¹H-NMR (CDCl₃, Varian, 400 MHz): δ 2.45 (3H, d, J=1.2 Hz), 3.36 (3H, s), 3.95 (3H, s), 6.40 (1H, t, J=8.4 Hz), 6.54 (1H, m), 7.34 (1H, d, J=8.4 Hz), 7.57 (1H, m), 9.72 (1H, s).

### Step C: potassium 6-(2-fluoro-4-iodophenylamino)-2,5-dimethyl-4-oxo-4,5-dihydrofuro[3,2-c]pyridine-7-carboxylate

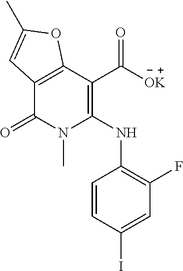

To a solution of methyl 6-(2-fluoro-4-iodophenylamino)-2,5-dimethyl-4-oxo-4,5-dihydrofuro[3,2-c]pyridine-7-carboxylate (273 mg, 0.599 mmol) in MeOH (18.0 mL) was added K₂CO₃ (331 mg, 2.40 mmol) at room temperature. And then Water (18.0 mL) was added slowly with dropping funnel. The reaction mixture was stirred at 70° C. for 1 hour. The reaction mixture was quenched with water and then concentrated in vacuo to give potassium 6-(2-fluoro-4-iodophenylamino)-2,5-dimethyl-4-oxo-4,5-dihydrofuro[3,2-c]pyridine-7-carboxylate (554 mg, 192%) as a white solid. LC-MS: Calcd. 441.98. Found 442.72 [M+H]⁺.

### Step D: 6-(2-fluoro-4-iodophenylamino)-2,5-dimethyl-4-oxo-N-(2-(vinyloxy)ethoxy)-4,5-dihydrofuro[3,2-c]pyridine-7-carboxamide

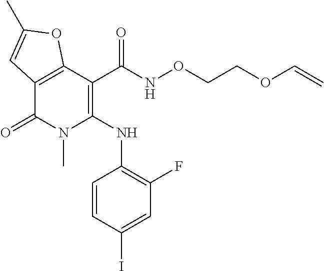

To a solution of potassium 6-(2-fluoro-4-iodophenylamino)-2,5-dimethyl-4-oxo-4,5-dihydrofuro[3,2-c]pyridine-7-carboxylate (280 mg, 0.583 mmol) in DMF (6.50 mL) was added O-(2-(vinyloxy)ethyl)hydroxylamine (intermediate 2, 66.1 mg, 0.641 mmol) at room temperature and then was cooled to 0° C. To a reaction mixture was added EDCI (168 mg, 0.875 mmol), HOBt (134 mg, 0.875 mmol) and TEA (0.143 mL, 1.02 mmol). The mixture was stirred at 40° C. for 15 h. The residue was extracted with EtOAc and washed with water and brine, dried over Na₂SO₄, filtered and concentrated in vacuo. The residue was purified by column chromatography on SiO₂ (Hex:EtOAc=2:1) to give 6-(2-fluoro-4-iodophenylamino)-2,5-dimethyl-4-oxo-N-(2-(vinyloxy)ethoxy)-4,5-dihydrofuro[3,2-c]pyridine-7-carboxamide (42.3 mg, 14%) as a brown solid. ¹H-NMR (CDCl₃, Varian, 400 MHz): δ 2.46 (3H, s), 3.32 (3H, s), 4.00 (2H, m), 4.07-4.27 (2H, m), 4.29-4.31 (2H, m), 6.48-6.55 (2H, m), 7.36 (1H, d, J=12.8 Hz), 7.46 (1H, dd, J=10.0, 2.0 Hz), 7.70 (1H, d, J=8.0 Hz), 10.0 (1H, s), 10.8 (1H, s).

### Step E: 6-(2-fluoro-4-iodophenylamino)-N-(2-hydroxyethoxy)-2,5-dimethyl-4-oxo-4,5-dihydrofuro[3,2-c]pyridine-7-carboxamide

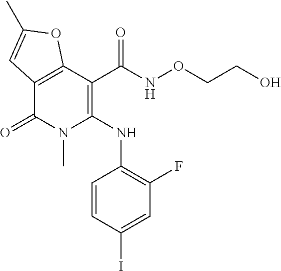

To a solution of 6-(2-fluoro-4-iodophenylamino)-2,5-dimethyl-4-oxo-N-(2-(vinyloxy)ethoxy)-4,5-dihydrofuro[3,2-c]pyridine-7-carboxamide (42.3 mg, 0.0800 mmol) in MeOH (0.400 mL) was added 1 N aq. HCl (0.52 mL, 0.515 mmol) at room temperature. The reaction mixture was stirred at room temperature for 10 min. The solvent was evaporated, diluted with DCM, and aq. NaHCO₃ was added until pH 7 at 0° C. DCM was evaporated and solidify with ether/hexane to give 6-(2-fluoro-4-iodophenylamino)-N-(2-hydroxyethoxy)-2,5-dimethyl-4-oxo-4,5-dihydrofuro[3,2-c]pyridine-7-carboxamide (15.2 mg, 37.8%) as a light brown solid. ¹H-NMR (CDCl₃, Varian, 400 MHz): δ 2.49 (3H, s), 3.30 (3H, s), 3.77 (2H, m), 4.06 (1H, t, J=6.4 Hz), 4.11 (2H, t, J=4.4 Hz), 6.50 (1H, t, J=8.6 Hz), 7.58 (1H, m), 7.39 (1H, d, J=4.0 Hz), 7.48 (1H, d, J=9.8, 1.8 Hz), 9.84 (1H, s), 10.7 (1H, s). m/z=501.8 [M+H]⁺.

### Example 4

### 6-(2-fluoro-4-iodophenylamino)-N-(2-hydroxyethoxy)-3,5-dimethyl-4-oxo-4,5-dihydrofuro[3,2-c]pyridine-7-carboxamide

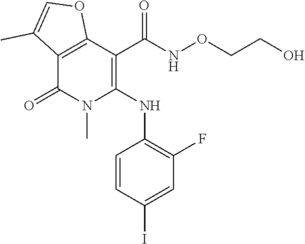

### Step A: ethyl 2-(2-ethoxy-2-oxoethyl)-4-methylfuran-3-carboxylate

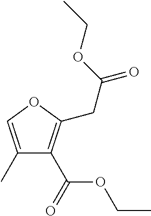

A mixture of Pd₂(dba)₃ (0.239 g, 0.261 mmol) and 1,2-Bis(diphenylphosphino)ethane (0.208 g, 0.521 mmol) in THF (226 mL) was added methyl prop-2-ynyl carbonate (intermediate 5, 5.94 g, 52.1 mmol) and diethyl 3-oxopentanedioate (10.5 g, 52.1 mmol) at room temperature. The reaction mixture was heated to reflux overnight under nitrogen atmosphere. After being cooled to room temperature, the reaction mixture was filtered through a Celite pad and washed with EtOAc. The filtrate was concentrated in vacuo, and the residue was purified by column chromatography on SiO₂ (Hex: EtOAc=4:1) to give ethyl 2-(2-ethoxy-2-oxoethyl)-4-methylfuran-3-carboxylate (4.67 g, 37%) as a yellow oil. ¹H-NMR (CDCl₃, Varian, 400 MHz): δ 1.26 (3H, t, J=9.0 Hz), 1.34 (3H, t, J=6.0 Hz), 2.16 (3H, s), 4.01 (2H, s), 4.18 (2H, q), 4.28 (2H, q), 7.12 (1H, s).

### Step B: ethyl 6-(2-fluoro-4-iodophenylamino)-3,5-dimethyl-4-oxo-4,5-dihydrofuro[3,2-c]pyridine-7-carboxylate

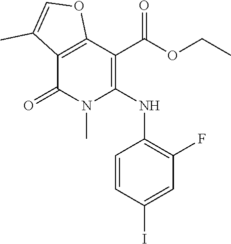

To a solution of ethyl 2-(2-ethoxy-2-oxoethyl)-4-methylfuran-3-carboxylate (4.67 g, 19.4 mmol) in dry THF (100 mL) was added NaH (55 wt % dispersion in mineral oil, 0.891 g, 20.4 mmol) at 0° C. The reaction mixture was stirred at room temperature for 30 min, and then 2-fluoro-4-iodo-N-((methylimino)methylene)aniline (intermediate 1, 6.98 g, 25.3 mmol) was added slowly. The reaction mixture was stirred at room temperature for 15 hours. The reaction mixture was quenched with water, extracted with EtOAc. The resulting solid was collected by filtration, rinsed with water and dried in vacuo to give ethyl 6-(2-fluoro-4-iodophenylamino)-3,5-dimethyl-4-oxo-4,5-dihydrofuro[3,2-c]pyridine-7-carboxylate (1.22 g, 13%) as a yellow solid, which was used for the next reaction without further purification. ¹H-NMR (CDCl₃, Varian, 400 MHz): δ 1.41 (3H, t, J=7.2 Hz), 2.36 (3H, s), 3.33 (3H, s), 4.39 (2H, q), 6.43 (1H, t, J=8.4 Hz), 7.27 (1H, m), 7.35 (1H, m), 7.48 (1H, m), 9.78 (1H, s).

### Step C: 6-(2-fluoro-4-iodophenylamino)-3,5-dimethyl-4-oxo-4,5-dihydrofuro[3,2-c]pyridine-7-carboxylic acid

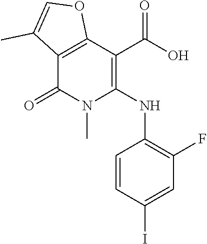

To a solution of ethyl 6-(2-fluoro-4-iodophenylamino)-3,5-dimethyl-4-oxo-4,5-dihydrofuro[3,2-c]pyridine-7-carboxylate (1.22 g, 2.59 mmol) in THF (12.0 mL) and MeOH (12.0 mL) was added K₂CO₃ (1.43 g, 10.4 mmol) at room temperature. And then Water (12.0 mL) was added slowly with dropping funnel. The reaction mixture was stirred at 65° C. for 9 hours. The reaction mixture was quenched with water and then acidified with 1N aq. HCl until pH 1˜2. The resulting solid was collected by filtration, rinsed with water and dried in vacuo to give 6-(2-fluoro-4-iodophenylamino)-3,5-dimethyl-4-oxo-4,5-dihydrofuro[3,2-c]pyridine-7-carboxylic acid (860 mg, 75%) as a white solid, which was used for the next reaction without further purification. ¹H-NMR (CDCl₃, Varian, 400 MHz): δ 2.21 (3H, s), 3.19 (3H, s), 6.64 (1H, t, J=8.2 Hz), 7.37 (1H, m), 7.60 (1H, m), 7.65 (1H, m), 9.59 (1H, s).

### Step D: 6-(2-fluoro-4-iodophenylamino)-3,5-dimethyl-4-oxo-N-(2-(vinyloxy)ethoxy)-4,5-dihydrofuro[3,2-c]pyridine-7-carboxamide

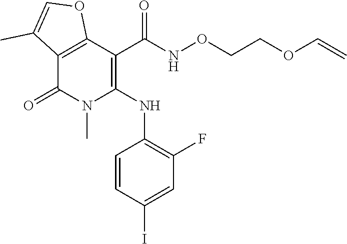

To a solution of 6-(2-fluoro-4-iodophenylamino)-3,5-dimethyl-4-oxo-4,5-dihydrofuro[3,2-c]pyridine-7-carboxylic acid (680 mg, 1.54 mmol) in DMF (35.0 mL) was added O-(2-(vinyloxy)ethyl)hydroxylamine (intermediate 2, 190 mg, 1.85 mmol) at room temperature and then was cooled to 0° C. To a reaction mixture was added EDCI (442 mg, 2.31 mmol), HOBt (353 mg, 2.31 mmol) and TEA (0.322 mL, 2.31 mmol). The mixture was stirred at room temperature for 15 hours. The residue was extracted with EtOAc and washed with water and brine, dried over Na₂SO₄, filtered and concentrated in vacuo. The residue was purified by column chromatography on SiO₂ (Hex: EtOAc=2:1) to give 6-(2-fluoro-4-iodophenylamino)-3,5-dimethyl-4-oxo-N-(2-(vinyloxy)ethoxy)-4,5-dihydrofuro[3,2-c]pyridine-7-carboxamide (382 mg, 47%) as a brown solid. ¹H-NMR (CDCl₃, Varian, 400 MHz): δ 2.37 (3H, s), 3.30 (3H, s), 4.00 (2H, m), 4.09 (1H, m), 4.22 (1H, m), 4.29 (2H, m), 6.49 (2H, m), 7.27 (1H, m), 7.37 (1H, m), 7.47 (1H, m), 10.0 (1H, s), 10.9 (1H, s).

### Step E: 6-(2-fluoro-4-iodophenylamino)-N-(2-hydroxyethoxy)-3,5-dimethyl-4-oxo-4,5-dihydrofuro[3,2-c]pyridine-7-carboxamide

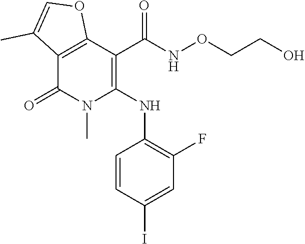

To a solution of 6-(2-fluoro-4-iodophenylamino)-3,5-dimethyl-4-oxo-N-(2-(vinyloxy)ethoxy)-4,5-dihydrofuro[3,2-c]pyridine-7-carboxamide (200 mg, 0.379 mmol) in MeOH (11.1 mL) and THF (11.1 mL) was added 1N aq. HCl (2.20 mL, 2.20 mmol) at room temperature. The reaction mixture was stirred at room temperature for 10 min. The reaction mixture was added aq. NaHCO₃ until pH 7 at 0° C. The residue was extracted with EtOAc and washed with water and brine, dried over Na₂SO₄, filtered and concentrated in vacuo. The residue was purified by column chromatography on SiO₂ (Hex:EtOAc=1:1) to give 6-(2-fluoro-4-iodophenylamino)-N-(2-hydroxyethoxy)-3,5-dimethyl-4-oxo-4,5-dihydrofuro[3,2-c]pyridine-7-carboxamide (84.1 mg, 44%) as a light brown solid. ¹H-NMR (CDCl₃, Varian, 400 MHz): δ 2.26 (3H, s), 3.34 (3H, s), 3.48 (2H, t, J=4.6 Hz), 3.65 (2H, t, J=4.8 Hz), 6.53 (1H, t, J=8.8 Hz), 7.33 (1H, m), 7.58 (1H, dd, J=10.8, 1.6 Hz), 7.64 (1H, m). * OH alcohol, NH amide, NH peak were not observed. m/z=501.8 [M+H]⁺.

### Example 5

### 6-(2-fluoro-4-iodophenylamino)-N-(2-hydroxyethoxy)-5-methyl-4-oxo-4,5-dihydrothieno[3,2-c]pyridine-7-carboxamide

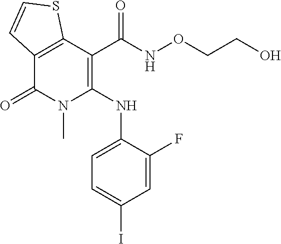

### Step A: methyl 2-(2-methoxy-2-oxoethyl)thiophene-3-carboxylate

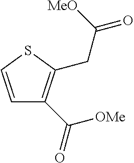

A mixture of Dimethyl 3-oxopentanedioate (13.7 g, 79.0 mmol), 1,4-dithiane-2,5-diol (4.00 g, 26.3 mmol), LiBr (685 mg, 7.88 mmol) in 1,4-dioxane (132 mL) was refluxed overnight, cooled to room temperature. The insoluble solid was filtered and washed with Et₂O, and concentrated in vacuo. The residue was purified by column chromatography on SiO₂ (Hex:EtOAc=85:15) to give the methyl 2-(2-methoxy-2-oxoethyl)thiophene-3-carboxylate (5.07 g, 90%) as a yellow oil. ¹H NMR (CDCl₃, Varian 400 MHz): δ 3.74 (3H, s), 3.84 (3H, s), 4.22 (2H, s), 7.15 (1H, d, J=5.2 Hz), 7.44 (1H, d, J=5.2 Hz).

### Step B: methyl 6-(2-fluoro-4-iodophenylamino)-5-methyl-4-oxo-4,5-dihydrothieno[3,2-c]pyridine-7-carboxylate

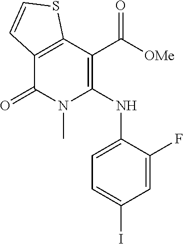

To a solution of methyl 2-(2-methoxy-2-oxoethyl)thiophene-3-carboxylate (1.00 g, 4.67 mmol) in dry THF (23.0 mL) was added NaH (55%, 224 mg, 5.13 mmol) at 0° C. The mixture was stirred for 30 minutes at 0° C. After dropwise addition of 2-fluoro-4-iodo-N-((methylimino)methylene)aniline (intermediate 1, 1.29 g, 4.67 mmol) at 0° C., the reaction mixture was stirred at room temperature for 30 min, and then quenched saturated aq. NH₄Cl (30 mL). The mixture was extracted with EtOAc (2×20 mL). The combined organic layers were washed water (50 mL) and brine (50 mL), dried over Na₂SO₄, filtered and concentrated in vacuo. The residue was purified by column chromatography on SiO₂ (Hex:EtOAc=4:1) to give the methyl 6-(2-fluoro-4-iodophenylamino)-5-methyl-4-oxo-4,5-dihydrothieno[3,2-c]pyridine-7-carboxylate (654 mg, 31%) as a yellow solid. ¹H NMR (CDCl₃, Varian 400 MHz): δ 3.38 (3H, s), 3.97 (3H, s), 6.45 (1H, t, J=8.4 Hz), 7.19 (1H, d, J=5.2 Hz), 7.36 (1H, d, J=8.4 Hz), 7.49 (1H, dd, J=10.0, 2.0 Hz), 7.61 (1H, d, J=8.4 Hz), 9.70 (1H, brs).

### Step C: 6-(2-fluoro-4-iodophenylamino)-5-methyl-4-oxo-N-(2-(vinyloxy)ethoxy)-4,5-dihydrothieno[3,2-c]pyridine-7-carboxamide

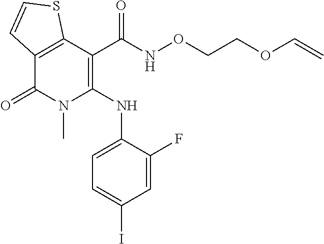

A mixture of methyl 6-(2-fluoro-4-iodophenylamino)-5-methyl-4-oxo-4,5-dihydrothieno[3,2-c]pyridine-7-carboxylate (1.36 g, 2.96 mmol) and O-(2-(vinyloxy)ethyl)hydroxylamine (intermediate 2, 458 mg, 4.44 mmol) in dry THF (20 mL) was added LiHMDS (17.8 mL, 17.8 mmol, 1.0 M solution in hexane) at 0° C. The reaction mixture was stirred for 1 hour at room temperature, and then quenched saturated 1N aq. HCl (50 mL). The mixture was extracted with EtOAc (2×20 mL). The combined organic layers were washed water (50 mL) and brine (50 mL), dried over Na₂SO₄, filtered and concentrated in vacuo. The residue was purified by column chromatography on SiO₂ (Hex:EtOAc=3:7) to give the 6-(2-fluoro-4-iodophenylamino)-5-methyl-4-oxo-N-(2-(vinyloxy)ethoxy)-4,5-dihydrothieno[3,2-c]pyridine-7-carboxamide (575 mg, 37%) as a yellow solid. ¹H NMR (CDCl₃, Varian 400 MHz): δ 3.41 (3H, s), 3.94-3.97 (2H, m), 4.07 (1H, dd, J=6.8, 2.4 Hz), 4.18-4.25 (3H, m), 6.37 (1H, t, J=8.4 Hz), 6.46 (1H, dd, J=14.6, 6.8 Hz), 7.28 (1H, d, J=5.2 Hz), 7.32 (1H, d, J=8.4 Hz), 7.47 (1H, dd, J=10.0, 2.0 Hz), 7.62 (1H, d, J=5.2 Hz), 8.71 (1H, brs), 9.21 (1H, brs).

### Step D: 6-(2-fluoro-4-iodophenylamino)-N-(2-hydroxyethoxy)-5-methyl-4-oxo-4,5-dihydrothieno[3,2-c]pyridine-7-carboxamide

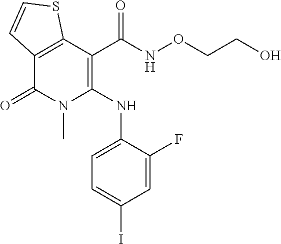

To a solution of 6-(2-fluoro-4-iodophenylamino)-5-methyl-4-oxo-N-(2-(vinyloxy)ethoxy)-4,5-dihydrothieno[3,2-c]pyridine-7-carboxamide (300 mg, 0.567 mmol) in MeOH (6.0 mL) was added 2N aq. HCl (1.7 mL, 3.40 mmol) at room temperature, the reaction mixture was stirred for 30 minutes at room temperature, then concentrated in vacuo. The residue was dissolved in DCM, neutralized with saturated aq. NaHCO₃ at 0° C. The separated aqueous layer was extracted with DCM. The combined organic layers were washed with brine, dried over Na₂SO₄, filtered and concentrated in vacuo. The residual solid was suspended in MeOH, collected by filtration, and washed with MeOH to give the 6-(2-fluoro-4-iodophenylamino)-N-(2-hydroxyethoxy)-5-methyl-4-oxo-4,5-dihydrothieno[3,2-c]pyridine-7-carboxamide (217 mg, 76%) as a white solid. ¹H NMR (DMSO-d₆, Varian 400 MHz): δ 3.43 (3H, s), 3.40-3.47 (2H, m), 3.57-3.64 (2H, m), 4.67 (1H, brs), 6.51 (1H, t, J=8.8 Hz), 7.31 (1H, dd, J=8.4, 1.2 Hz), 7.49 (1H, d, J=5.2 Hz), 7.57 (1H, dd, J=10.8, 2.0 Hz), 7.62 (1H, d, J=5.2 Hz), 8.30 (1H, brs), 11.30 (1H, brs). m/z=503.7 [M+H]⁺.

### Example 6

### 6-(2-fluoro-4-iodophenylamino)-N-(2-hydroxyethoxy)-2,5-dimethyl-4-oxo-4,5-dihydrooxazolo[4,5-c]pyridine-7-carboxamide

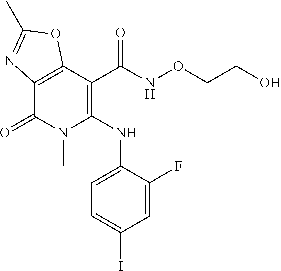

### Step A: dimethyl 2-(hydroxyimino)-3-oxopentanedioate

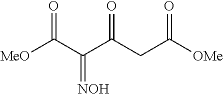

To a solution of dimethyl 3-oxopentanedioate (25.0 g, 144 mmol) in acetic acid (50 mL) was added a solution of NaNO₂ (10.4 g, 151 mmol) in water (25 mL) at 0° C. The reaction mixture was stirred overnight at room temperature. After evaporation of volatile solvents, the residue was partitioned between EtOAc (50 mL) and water (50 mL). The separated aqueous layer was extracted (2×30 mL). The combined organic layers were washed water (100 mL) and brine (100 mL), dried over Na₂SO₄, filtered and concentrated in vacuo. The residue was purified by column chromatography on SiO₂ (Hex:EtOAc=3:2) to give the dimethyl 2-(hydroxyimino)-3-oxopentanedioate (15.3 g, 52%) as a yellow oil. ¹H NMR (CDCl₃, Varian 400 MHz): δ 3.76 (3H, s), 3.84 (2H, s), 3.93 (3H, s), 9.74 (1H, brs).

### Step B: dimethyl 2-acetamido-3-oxopentanedioate

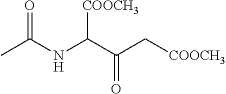

A mixture of dimethyl 2-(hydroxyimino)-3-oxopentanedioate (15.3 g, 75.0 mmol) and 10% Pd/C (1.50 g) in Acetic anhydride (377 mL) was stirred at room temperature for 3 hours under hydrogen atmosphere (balloon). The reaction mixture was filtered through a Celite pad, washed with EtOAc (200 mL), and concentrated in vacuo. The residue was purified by column chromatography on SiO₂ (Hex:EtOAc=1:9) to give the dimethyl 2-acetamido-3-oxopentanedioate (9.28 g, 53%) as a yellow oil. ¹H NMR (CDCl₃, Varian 400 MHz): δ 2.09 (1H, s), 3.75 (3H, s), 3.76 (2H, s), 3.83 (3H, s), 5.42 (1H, d, J=6.4 Hz), 6.64 (1H, brs).

### Step C: methyl 5-(2-methoxy-2-oxoethyl)-2-methyloxazole-4-carboxylate

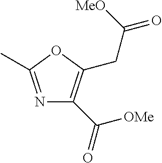

To a solution of dimethyl 2-acetamido-3-oxopentanedioate (9.28 g, 40.2 mmol) in chloroform (200 mL) was added SOCl₂ (8.79 mL, 120 mmol) at 0° C. The mixture was refluxed for 6 hours, cooled to room temperature. After evaporation of volatile solvents, the residue was diluted with DCM (50 mL), neutralized with saturated aq. NaHCO₃ at 0° C., and extracted with DCM (2×30 mL). The combined organic layers were washed water (100 mL) and brine (100 mL), dried over Na₂SO₄, filtered and concentrated in vacuo. The residue was purified by column chromatography on SiO₂ (Hex:EtOAc=1:2) to give the methyl 5-(2-methoxy-2-oxoethyl)-2-methyloxazole-4-carboxylate (4.06 g, 47%) as a yellow solid. ¹H NMR (CDCl₃, Varian 400 MHz): δ 2.49 (3H, s), 3.75 (3H, s), 3.90 (3H, s), 4.10 (2H, s).

### Step D: methyl 6-(2-fluoro-4-iodophenylamino)-2,5-dimethyl-4-oxo-4,5-dihydrooxazolo[4,5-c]pyridine-7-carboxylate

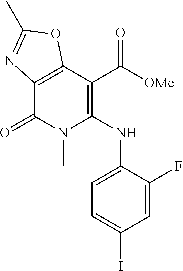

To a solution of methyl 5-(2-methoxy-2-oxoethyl)-2-methyloxazole-4-carboxylate (2.00 g, 4.67 mmol) in dry THF (47 mL) was added NaH (55%, 450 mg, 10.3 mmol) at 0° C. The mixture was stirred for 30 minutes at 0° C. After dropwise addition of 2-fluoro-4-iodo-N-((methylimino)methylene)aniline (intermediate 1, 3.11 g, 11.3 mmol) at 0° C., the reaction mixture was stirred at 0° C. for 30 min, and then quenched saturated aq. NH₄Cl (50 mL). The mixture was extracted with EtOAc (2×30 mL). The combined organic layers were washed water (70 mL) and brine (70 mL), dried over Na₂SO₄, filtered and concentrated in vacuo. The residual was suspended in Et₂O, collected by filtration, and washed with Et₂O to give the methyl 6-(2-fluoro-4-iodophenylamino)-2,5-dimethyl-4-oxo-4,5-dihydrooxazolo[4,5-c]pyridine-7-carboxylate (1.85 g, 43%) as a brown solid. ¹H NMR (CDCl₃, Varian 400 MHz): δ 2.64 (3H, s), 3.36 (3H, s), 3.96 (3H, s), 6.50 (1H, t, J=8.4 Hz), 7.40 (1H, d, J=8.4 Hz), 7.50 (1H, dd, J=10.0, 2.0 Hz), 9.92 (1H, brs).

### Step E: 6-(2-fluoro-4-iodophenylamino)-2,5-dimethyl-4-oxo-4,5-dihydrooxazolo[4,5-c]pyridine-7-carboxylic acid

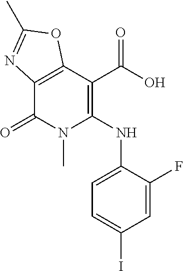

To a solution of methyl 6-(2-fluoro-4-iodophenylamino)-2,5-dimethyl-4-oxo-4,5-dihydrooxazolo[4,5-c]pyridine-7-carboxylate (1.60 g, 3.50 mmol) in a mixture of THF (40 mL), MeOH (40 mL) and H₂O (40 mL) was added 2 M aq. K₂CO₃ (2.62 mL, 5.25 mmol) at room temperature. The reaction mixture was stirred at 70° C. for 3 hours. The mixture was extracted with EtOAc (2×40 mL). The aqueous layer was acidified with 3 N aq. HCl until pH 3. The resulting solid was collected by filtration, washed with water and Et₂O to give the 6-(2-fluoro-4-iodophenylamino)-2,5-dimethyl-4-oxo-4,5-dihydrooxazolo[4,5-c]pyridine-7-carboxylic acid (1.21 g, 78%) as a yellow solid. ¹H-NMR (DMSO-d₆, Varian, 400 MHz): δ 2.56 (3H, s), 3.25 (3H, s), 6.75 (1H, t, J=8.8 Hz), 7.44 (1H, d, J=8.8 Hz), 7.69 (1H, dd, J=10.8, 2.0 Hz), 9.70 (1H, s), 13.33 (1H, brs).

### Step F: 6-(2-fluoro-4-iodophenylamino)-2,5-dimethyl-4-oxo-N-(2-(vinyloxy)ethoxy)-4,5-dihydrooxazolo[4,5-c]pyridine-7-carboxamide

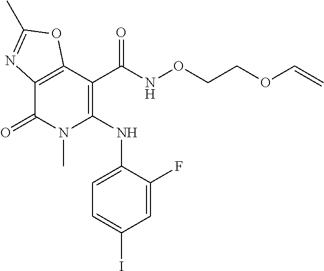

A mixture of 6-(2-fluoro-4-iodophenylamino)-2,5-dimethyl-4-oxo-4,5-dihydrooxazolo[4,5-c]pyridine-7-carboxylic acid (1.21 g, 2.74 mmol), O-(2-(vinyloxy)ethyl)hydroxylamine (intermediate 2, 424 mg, 4.11 mmol), HOBT (630 mg, 4.11 mmol), and EDC (788 mg, 4.11 mmol) in DMF (14 mL) was added TEA (0.764 mL, 5.48 mmol) at room temperature. The mixture was stirred at room temperature for 1 h, and quenched with saturated aq. NH₄Cl (20 mL). The mixture was extracted with EtOAc (3×10 mL), and the combined organic layers were washed with water (3×30 mL) and brine, dried over Na₂SO₄, filtered and concentrated in vacuo. The residual was suspended in EtOAc, collected by filtration, and washed with EtOAc to give the 6-(2-fluoro-4-iodophenylamino)-2,5-dimethyl-4-oxo-N-(2-(vinyloxy)ethoxy)-4,5-dihydrooxazolo[4,5-c]pyridine-7-carboxamide (826 g, 57%) as a yellow solid. ¹H-NMR (CDCl₃, Varian, 400 MHz): δ 2.66 (3H, s), 3.33 (3H, s), 4.00-4.02 (2H, m), 4.09-4.12 (1H, m), 4.26 (1H, dd, J=14.4, 2.4 Hz), 4.30-4.33 (2H, m), 6.50-6.57 (2H, m), 7.40 (1H, d, J=8.4 Hz), 7.48 (1H, dd, J=10.0, 2.0 Hz), 9.70 (1H, s), 10.96 (1H, s).

### Step G: 6-(2-fluoro-4-iodophenylamino)-N-(2-hydroxyethoxy)-2,5-dimethyl-4-oxo-4,5-dihydrooxazolo[4,5-c]pyridine-7-carboxamide

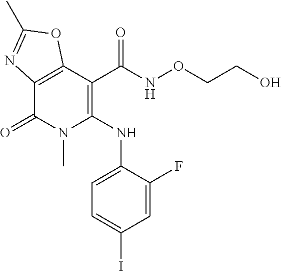

To a solution of 6-(2-fluoro-4-iodophenylamino)-2,5-dimethyl-4-oxo-N-(2-(vinyloxy)ethoxy)-4,5-dihydrooxazolo[4,5-c]pyridine-7-carboxamide (300 mg, 0.568 mmol) in MeOH (6.0 mL) was added 2N aq. HCl (1.7 mL, 3.40 mmol) at room temperature, the reaction mixture was stirred for 30 minutes at room temperature, then concentrated in vacuo. The residue was dissolved in DCM, neutralized with saturated aq. NaHCO₃ at 0° C. The separated aqueous layer was extracted with DCM. The combined organic layers were washed with brine, dried over Na₂SO₄, filtered and concentrated in vacuo. The residue was purified by column chromatography on SiO₂ (DCM:MeOH=95:5) to give the 6-(2-fluoro-4-iodophenylamino)-N-(2-hydroxyethoxy)-2,5-dimethyl-4-oxo-4,5-dihydrooxazolo[4,5-c]pyridine-7-carboxamide (186 mg, 65%) as a white solid. ¹H NMR (DMSO-d₆, Varian 400 MHz): δ 2.56 (3H, s), 3.39 (3H, s), 3.47-3.50 (2H, m), 3.59-3.62 (2H, m), 4.68 (1H, t, J=5.6 Hz), 6.63 (1H, t, J=8.8 Hz), 7.35 (1H, d, J=8.4 Hz), 7.60 (1H, dd, J=10.8, 2.0 Hz), 9.00 (1H, brs), 11.34 (1H, brs). m/z=502.9 [M+H]⁺.

### Example 7

### 6-(2-fluoro-4-iodophenylamino)-N-(2-hydroxyethoxy)-5-methyl-4-oxo-4,5-dihydrothiazolo[4,5-c]pyridine-7-carboxamide

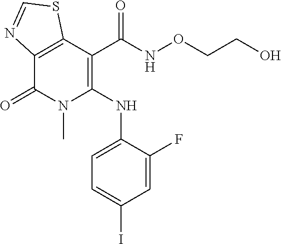

### Step A: dimethyl 3-bromo-2-oxopentanedioate

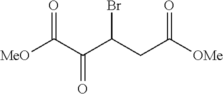

To a solution of compound CuBr₂ (11.5 g, 51.7 mmol) in EtOAc (160 mL) was added a solution of Dimethyl 2-oxopentanedioate (3.00 g, 17.2 mmol) in CHCl₃ (80 mL) at room temperature. The reaction mixture was refluxed overnight, cooled to room temperature. After evaporation of volatile solvents, the residue was purified by column chromatography on SiO₂ (Hex:EtOAc=3:2) to give the dimethyl 3-bromo-2-oxopentanedioate (4.36 g, quant.) as a yellow oil. ¹H NMR (CDCl₃, Varian 400 MHz): δ 3.06 (1H, ABX, J_{ab}=17.3 Hz, Jₐₓ=9.3 Hz), 3.34 (1H, ABX, J_{ab}=17.3 Hz, J_{bx}=5.9 Hz), 3.71 (3H, s), 3.95 (3H, s), 5.40 (1H, ABX, Jₐₓ=9.3 Hz, J_{bx}=5.9 Hz).

### Step B: methyl 5-(2-methoxy-2-oxoethyl)thiazole-4-carboxylate

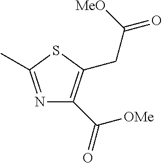

A mixture of dimethyl 3-bromo-2-oxopentanedioate (4.36 g, 17.2 mmol) and methanethioamide (1.6 M in dioxane, 53.8 mL, 86.0 mmol) in EtOH (86 mL) was refluxed overnight, cooled to room temperature. After evaporation of volatile solvents, the residue was purified by column chromatography on SiO₂ (Hex:EtOAc=2:3) to give the methyl 5-(2-methoxy-2-oxoethyl)thiazole-4-carboxylate (2.02 g, 54%) as a yellow oil. ¹H NMR (CDCl₃, Varian 400 MHz): δ 3.77 (3H, s), 3.96 (3H, s), 4.37 (2H, s), 8.73 (1H, s).

### Step C: methyl 6-(2-fluoro-4-iodophenylamino)-5-methyl-4-oxo-4,5-dihydrothiazolo[4,5-c]pyridine-7-carboxylate

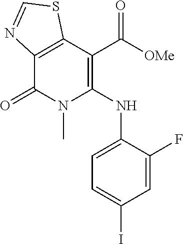

To a solution of methyl 5-(2-methoxy-2-oxoethyl)thiazole-4-carboxylate (2.02 g, 9.40 mmol) in dry THF (47 mL) was added NaH (55%, 490 mg, 11.3 mmol) at 0° C. The mixture was stirred for 30 minutes at 0° C. After dropwise addition of 2-fluoro-4-iodo-N-((methylimino)methylene)aniline (intermediate 1, 2.59 g, 9.40 mmol) at 0° C., the reaction mixture was stirred at room temperature for 1 hour, and then quenched saturated aq. NH₄Cl (50 mL). The mixture was extracted with EtOAc (2×30 mL). The combined organic layers were washed water (70 mL) and brine (70 mL), dried over Na₂SO₄, filtered and concentrated in vacuo. The residual was purified by column chromatography on SiO₂ (Hex:EtOAc=2:3 to 1:2) to give the methyl 6-(2-fluoro-4-iodophenylamino)-5-methyl-4-oxo-4,5-dihydrothiazolo[4,5-c]pyridine-7-carboxylate (434 mg, 10%) as an orange solid. ¹H NMR (CDCl₃, Varian 400 MHz): δ 3.41 (3H, s), 3.97 (3H, s), 6.54 (1H, t, J=8.8 Hz), 7.41 (1H, d, J=8.4 Hz), 7.52 (1H, dd, J=10.0, 2.0 Hz), 9.94 (1H, brs).

### Step D: 6-(2-fluoro-4-iodophenylamino)-5-methyl-4-oxo-4,5-dihydrothiazolo[4,5-c]pyridine-7-carboxylic acid

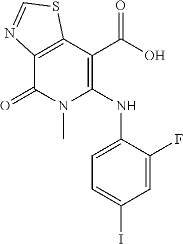

To a solution of methyl 6-(2-fluoro-4-iodophenylamino)-5-methyl-4-oxo-4,5-dihydrothiazolo[4,5-c]pyridine-7-carboxylate (100 mg, 0.218 mmol) in a mixture of THF (2 mL), MeOH (2 mL) and H₂O (2 mL) was added 2 M aq. K₂CO₃ (0.163 mL, 0.327 mmol) at room temperature. The reaction mixture was stirred at 70° C. for 3 hours. The mixture was extracted with EtOAc (2×10 mL). The aqueous layer was acidified with 3 N aq. HCl until pH 3. The resulting solid was collected by filtration, washed with water and Et₂O to give the 6-(2-fluoro-4-iodophenylamino)-5-methyl-4-oxo-4,5-dihydrothiazolo[4,5-c]pyridine-7-carboxylic acid (66.9 mg, 69%) as a yellow solid. ¹H-NMR (DMSO-d₆, Varian, 400 MHz): δ 3.28 (3H, s), 6.75 (1H, t, J=8.8 Hz), 7.40 (1H, d, J=8.4 Hz), 7.69 (1H, dd, J=10.6, 2.0 Hz), 9.07 (1H, s), 9.62 (1H, brs).

### Step E: 6-(2-fluoro-4-iodophenylamino)-5-methyl-4-oxo-N-(2-(vinyloxy)ethoxy)-4,5-dihydrothiazolo[4,5-c]pyridine-7-carboxamide

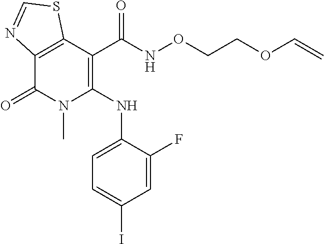

A mixture of 6-(2-fluoro-4-iodophenylamino)-5-methyl-4-oxo-4,5-dihydrothiazolo[4,5-c]pyridine-7-carboxylic acid (66.9 mg, 0.150 mmol), O-(2-(vinyloxy)ethyl)hydroxylamine (intermediate 2, 23.0 mg, 0.225 mmol) in DMF (1 mL) was added HATU (171 mg, 0.450 mmol) at room temperature. The mixture was stirred at room temperature for 4 hours, and quenched with saturated aq. NH₄Cl (10 mL). The mixture was extracted with EtOAc (2×10 mL), and the combined organic layers were washed with water (3×10 mL) and brine, dried over Na₂SO₄, filtered and concentrated in vacuo. The residual was purified by column chromatography on SiO₂ (DCM:MeOH=95:5) to give the 6-(2-fluoro-4-iodophenylamino)-5-methyl-4-oxo-N-(2-(vinyloxy)ethoxy)-4,5-dihydrothiazolo[4,5-c]pyridine-7-carboxamide (14 mg, 17%) as a brown solid. ¹H-NMR (CDCl₃, Varian, 400 MHz): δ 3.69 (3H, s), 3.90-3.93 (2H, m), 3.98-4.02 (3H, m), 4.13 (1H, dd, J=14.4, 2.4 Hz), 6.47-6.52 (2H, m), 7.31 (1H, d, J=8.8 Hz), 7.43 (1H, dd, J=10.0, 2.0 Hz), 8.90 (1H, s).

### Step F: 6-(2-fluoro-4-iodophenylamino)-N-(2-hydroxyethoxy)-5-methyl-4-oxo-4,5-dihydrothiazolo[4,5-c]pyridine-7-carboxamide

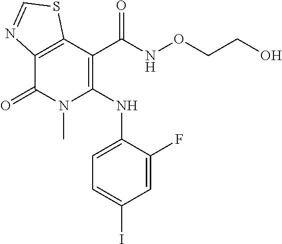

To a solution of 6-(2-fluoro-4-iodophenylamino)-5-methyl-4-oxo-N-(2-(vinyloxy)ethoxy)-4,5-dihydrothiazolo[4,5-c]pyridine-7-carboxamide (14.0 mg, 0.026 mmol) in MeOH (0.5 mL) was added 2N aq. HCl (0.079 mL, 0.158 mmol) at room temperature, the reaction mixture was stirred for 1 hour at room temperature, then concentrated in vacuo. The residue was purified by column chromatography on SiO₂ (DCM:MeOH=93:7 to 9:1) to give the 6-(2-fluoro-4-iodophenylamino)-N-(2-hydroxyethoxy)-5-methyl-4-oxo-4,5-dihydrothiazolo[4,5-c]pyridine-7-carboxamide (5.2 mg, 39%) as a yellow solid. ¹H NMR (CDCl₃, Varian 400 MHz): δ 3.40-3.47 (2H, m), 3.74 (3H, s), 3.76-3.81 (2H, m), 6.52 (1H, t, J=8.8 Hz), 7.34 (1H, d, J=8.4 Hz), 7.44 (1H, dd, J=10.0, 1.6 Hz), 8.94 (1H, s), 9.20 (1H, brs), 11.06 (1H, brs). m/z=504.6 [M+H]⁺.

### Example 8

### 6-(2-chloro-4-iodophenylamino)-N-(2-hydroxyethoxy)-5-methyl-4-oxo-4,5-dihydrofuro[3,2-c]pyridine-7-carboxamide

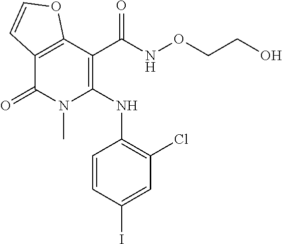

### Step A: 6-(2-chloro-4-iodophenylamino)-5-methyl-4-oxo-N-(2-(vinyloxy)ethoxy)-4,5-dihydrofuro[3,2-c]pyridine-7-carboxamide

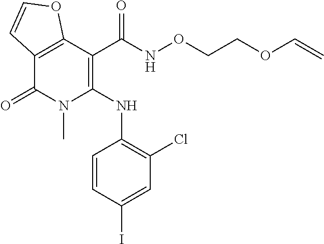

To a solution of methyl 6-(2-chloro-4-iodophenylamino)-5-methyl-4-oxo-4,5-dihydrofuro[3,2-c]pyridine-7-carboxylate (intermediate 7, 320 mg, 0.69 mmol) in dry THF (17.0 mL) was added O-(2-(vinyloxy)ethyl)hydroxylamine (intermediate 2, 108 mg, 1.04 mmol) at room temperature and then was cooled to 0° C. To the reaction mixture was added LiHMDS (3.50 g, 4.19 mmol) at 0° C. under a N₂ atmosphere. The mixture was stirred at room temperature for 1 hour. The reaction mixture was quenched with water and extracted with EtOAc, washed with water and brine, dried over Na₂SO₄, filtered and concentrated in vacuo to give 6-(2-chloro-4-iodophenylamino)-5-methyl-4-oxo-N-(2-(vinyloxy)ethoxy)-4,5-dihydrofuro[3,2-c]pyridine-7-carboxamide (310 mg, 84%) as a white solid. ¹H-NMR (CDCl₃, Varian, 400 MHz): δ 3.99-4.01 (2H, m), 4.07-4.09 (1H, dd, J=6.8, 2.4 Hz), 4.25-4.26 (1H, dd, J=14.4, 2.4 Hz), 6.36 (1H, d, J=8.4 Hz), 6.49-6.54 (1H, m), 7.01 (1H, m), 7.43-7.45 (1H, dd, J=8.6, 1.8 Hz), 7.51 (1H, m), 7.77 (1H, m), 10.0 (1H, s), 10.8 (1H, s).

### Step B: 6-(2-chloro-4-iodophenylamino)-N-(2-hydroxyethoxy)-5-methyl-4-oxo-4,5-dihydrofuro[3,2-c]pyridine-7-carboxamide

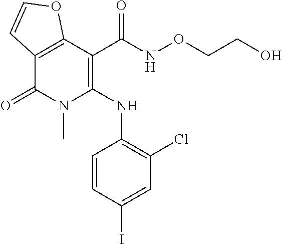

To a solution of 6-(2-chloro-4-iodophenylamino)-5-methyl-4-oxo-N-(2-(vinyloxy)ethoxy)-4,5-dihydrofuro[3,2-c]pyridine-7-carboxamide (200 mg, 0.37 mmol) in MeOH (4 mL) was added 2N aq. HCl (1.21 mL) at room temperature. The mixture was stirred at room temperature for 30 min. The residue was neutralized with aq. NaHCO₃ at 0° C. The separated aqueous layer was extracted with DCM. The combined organic layers were washed with brine, dried over Na₂SO₄, filtered and concentrated in vacuo. The residual solid was suspended in Et₂O, collected by filtration, and washed with Et₂O to give 6-(2-chloro-4-iodophenylamino)-N-(2-hydroxyethoxy)-5-methyl-4-oxo-4,5-dihydrofuro[3,2-c]pyridine-7-carboxamide (170 mg, 89%) as a white solid. ¹H-NMR (CDCl₃, Varian, 400 MHz) δ 3.29 (3H, s), 3.50-3.51 (2H, m), 3.70-3.72 (2H, m), 4.66-4.681 (1H, m), 6.46 (1H, t, J=8.4 Hz), 7.03 (1H, m), 7.45-7.48 (1H, dd, J=8.4, 2.0 Hz), 7.78 (1H, m), 7.96 (1H, m), 8.91 (1H, s), 11.3 (1H, s). m/z=503.6 [M+H]⁺.

### Example 9

### N-(cyclopropylmethoxy)-6-(2-fluoro-4-iodophenylamino)-5-methyl-4-oxo-4,5-dihydrofuro[3,2-c]pyridine-7-carboxamide

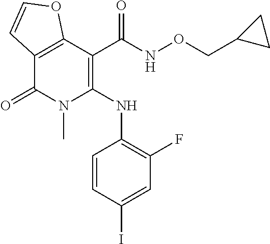

To a solution of 6-(2-fluoro-4-iodophenylamino)-5-methyl-4-oxo-4,5-dihydrofuro[3,2-c]pyridine-7-carboxylic acid (Example 1, Step A; 150 mg, 0.350 mmol) in DMF (1.00 mL) was added O-(cyclopropylmethyl)hydroxylamine hydrochloride (intermediate 8, 47.6 mg, 0.385 mmol) at room temperature and then was cooled to 0° C. To a reaction mixture was added EDCI (101 mg, 0.526 mmol), HOBt (80.0 mg, 0.526 mmol) and TEA (0.146 mL, 1.05 mmol). The mixture was stirred at room temperature for 15 hours. The residue was extracted with EtOAc and washed with water and brine, dried over Na₂SO₄, filtered and concentrated in vacuo. The residue was purified by column chromatography on SiO₂ (Hex: EtOAc=2:1) to give N-(cyclopropylmethoxy)-6-(2-fluoro-4-iodophenylamino)-5-methyl-4-oxo-4,5-dihydrofuro[3,2-c]pyridine-7-carboxamide (30.3 mg, 16%) as a purple solid. ¹H-NMR (CDCl₃, Varian, 400 MHz); δ 0.35 (2H, m), 0.63 (2H, m), 3.32 (3H, s), 3.87 (2H, d, J=7.2 Hz), 6.50 (1H, t, J=8.4 Hz), 7.01 (1H, m), 7.36 (1H, m), 7.47 (1H, m), 7.53 (1H, m), 9.86 (1H, s), 10.9 (1H, s). * NH peak was not observed. m/z=497.90 [M+H]⁺.

### Example 10

### 6-(2-fluoro-4-iodophenylamino)-N-(1-hydroxy-2-methylpropan-2-yloxy)-5-methyl-4-oxo-4,5-dihydrofuro[3,2-c]pyridine-7-carboxamide

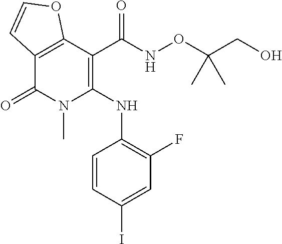

To a solution of 6-(2-fluoro-4-iodophenylamino)-5-methyl-4-oxo-4,5-dihydrofuro[3,2-c]pyridine-7-carboxylic acid (Example 1, Step A; 150 mg, 0.350 mmol) in DMF (3.50 mL) was added O-(1-hydroxy-2-methylpropan-2-yl)hydroxylammonium chloride (intermediate 9, 74.4 mg, 0.526 mmol) at room temperature and then was cooled to 0° C. To a reaction mixture was added EDCI (101 mg, 0.526 mmol), HOBt (80.0 mg, 0.526 mmol) and TEA (0.0730 mL, 0.526 mmol). The mixture was stirred at room temperature for 15 hours. The residue was extracted with EtOAc and washed with water and brine, dried over Na₂SO₄, filtered and concentrated in vacuo. The residue was purified by column chromatography on SiO₂(Hex:EtOAc=1:1) to give 6-(2-fluoro-4-iodophenylamino)-N-(1-hydroxy-2-methylpropan-2-yloxy)-5-methyl-4-oxo-4,5-dihydrofuro[3,2-c]pyridine-7-carboxamide (30.1 mg, 17%) as a white solid. ¹H-NMR (CDCl₃, Varian, 400 MHz): δ 1.34 (6H, s), 3.31 (3H, s), 3.39 (2H, d, J=6.8 Hz), 6.55 (1H, t, J=8.4 Hz), 7.02 (1H, d, J=2.0 Hz), 7.39 (1H, d, J=8.4 Hz), 7.48 (1H, m), 7.52 (1H, m), 9.46 (1H, s), 10.80 (1H, s). m/z=515.9.0 [M+H]⁺.

### Example 11

### 6-(2-fluoro-4-iodophenylamino)-N-methoxy-5-methyl-4-oxo-4,5-dihydrofuro[3,2-c]pyridine-7-carboxamide

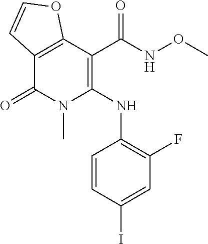

To a solution of 6-(2-fluoro-4-iodophenylamino)-5-methyl-4-oxo-4,5-dihydrofuro[3,2-c]pyridine-7-carboxylic acid (Example 1, Step A; 150 mg, 0.350 mmol) in DMF (3.50 mL) was added methoxymethanamine hydrochloride (51.3 mg, 0.526 mmol) at room temperature and then was cooled to 0° C. To a reaction mixture was added EDCI (101 mg, 0.526 mmol), HOBt (80.0 mg, 0.526 mmol) and TEA (0.0730 mL, 0.526 mmol). The mixture was stirred at room temperature for 15 hours. The residue was extracted with EtOAc and washed with water and brine, dried over Na₂SO₄, filtered and concentrated in vacuo. The residue was purified by column chromatography on SiO₂ (Hex: EtOAc=1:1) to give 6-(2-fluoro-4-iodophenylamino)-N-methoxy-5-methyl-4-oxo-4,5-dihydrofuro[3,2-c]pyridine-7-carboxamide (66.2 mg, 41%) as a purple solid. ¹H-NMR (CDCl₃, Varian, 400 MHz): δ 3.34 (3H, s), 3.90 (3H, s), 6.51 (1H, t, J=8.4 Hz), 7.01 (1H, s), 7.38 (1H, d, J=14 Hz), 7.49 (2H, m), 9.88 (1H, s), 10.98 (1H, s). m/z=458.0 [M+H]⁺.

### Example 12

### (R)—N-(2,3-dihydroxypropoxy)-6-(2-fluoro-4-iodophenylamino)-5-methyl-4-oxo-4,5-dihydrofuro[3,2-c]pyridine-7-carboxamide

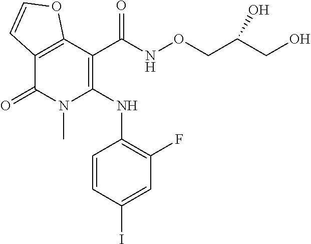

### Step A: (R)—N-((2,2-dimethyl-1,3-dioxolan-4-yl)methoxy)-6-(2-fluoro-4-iodophenylamino)-5-methyl-4-oxo-4,5-dihydrofuro[3,2-c]pyridine-7-carboxamide

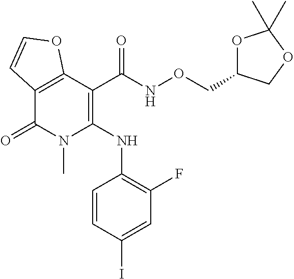

To a mixture of methyl 6-(2-fluoro-4-iodophenylamino)-5-methyl-4-oxo-4,5-dihydrofuro[3,2-c]pyridine-7-carb (Example 1, Step A; 1.30 g, 2.94 mmol) and (R)—O-((2,2-dimethyl-1,3-dioxolan-4-yl)methyl)hydroxylamine (intermediate 10, 0.65 g, 4.41 mmol in dry THF (7.35 mL) was added LiHMDS (17.6 mL, 16.6 mmol, 1.06 M solution in hexane) at 0° C. The reaction mixture was stirred for 20 min at 0° C., and then quenched with saturated 1N aq. HCl (50 mL). The mixture was extracted with EtOAc (3×20 mL). The combined organic layers were washed with water (50 mL) and brine (50 mL), dried over Na₂SO₄, filtered and concentrated in vacuo. The residue was purified by column chromatography on SiO₂ (Hex:EtOAc=1:1 to 1:2) to give (R)—N-((2,2-dimethyl-1,3-dioxolan-4-yl)methoxy)-6-(2-fluoro-4-iodophenylamino)-5-methyl-4-oxo-4,5-dihydrofuro[3,2-c]pyridine-7-carboxamide (1.07 g, 65.3%) as a yellow solid. ¹H NMR (CDCl₃, Varian 400 MHz) δ 1.40 (3H, s), 1.47 (3H, s), 3.32 (3H, s), 3.85 (1H, dd, J=8.4, 6.4 Hz), 4.05-4.18 (3H, m), 4.43-4.48 (1H, m), 6.51 (1H, t, J=8.4 Hz), 7.01 (1H, d, J=2.0 Hz), 7.37 (1H, d, J=8.4 Hz), 7.46-7.50 (2H, m), 10.14 (1H, s), 10.96 (1H, brs).

### Step B: (R)—N-(2,3-dihydroxypropoxy)-6-(2-fluoro-4-iodophenylamino)-5-methyl-4-oxo-4,5-dihydrofuro[3,2-c]pyridine-7-carboxamide

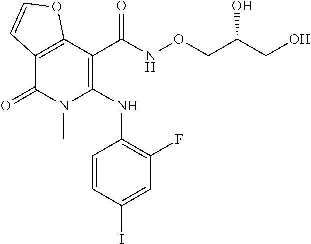

To a solution of (R)—N-((2,2-dimethyl-1,3-dioxolan-4-yl)methoxy)-6-(2-fluoro-4-iodophenylamino)-5-methyl-4-oxo-4,5-dihydrofuro[3,2-c]pyridine-7-carboxamide (1.07 g, 1.92 mmol) in MeOH (28 mL) was added 1 N aq. HCl (9.31 mL, 9.31 mmol) at room temperature. The mixture was stirred at room temperature for 14 hours. The residue was diluted with DCM and washed with water and brine, dried over Na₂SO₄, filtered and concentrated in vacuo to give (R)—N-(2,3-dihydroxypropoxy)-6-(2-fluoro-4-iodophenylamino)-5-methyl-4-oxo-4,5-dihydrofuro[3,2-c]pyridine-7-carboxamide (580 mg, 58.4%) as a white solid. ¹H-NMR (DMSO-d₆, Varian, 400 MHz) δ 2.42 (1H, t, J=5.8 Hz), 3.30 (3H, s), 3.62-3.67 (1H, m), 3.73-3.79 (1H, m), 3.97-4.02 (1H, m), 4.03-4.14 (2H, m), 4.44 (1H, d, t, J=2.0 Hz), 6.56 (1H, t, t, J=8.4 Hz), 7.01 (1H, d, t, J=2.0 Hz), 7.40 (1H, d, t, J=8.4 Hz), 7.49 (1H, dd, J=9.8, 1.8 Hz), 7.52 (1H, d, t, J=2.0 Hz), 10.00 (1H, s), 10.79 (1H, brs). m/z=517.8 [M+H]⁺.

### Example 13

### 6-(2-fluoro-4-iodophenylamino)-N-(3-hydroxypropyl)-5-methyl-4-oxo-4,5-dihydrofuro[3,2-c]pyridine-7-carboxamide

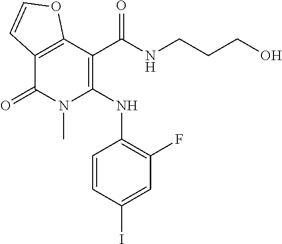

To a solution of 6-(2-fluoro-4-iodophenylamino)-5-methyl-4-oxo-4,5-dihydrofuro[3,2-c]pyridine-7-carboxylic acid (Example 1, Step A; 1.18 g, 2.76 mmol) in DMF (21.5 mL) was added 3-aminopropan-1-ol (228 mg, 3.03 mmol) at room temperature and then was cooled to 0° C. To the reaction mixture was added EDC (792 mg, 4.13 mmol), HOBT (633 g, 4.13 mmol), and TEA (0.77 ml, 5.51 mmol). The mixture was stirred at room temperature for 2 hours. The reaction was extracted with EtOAc, washed with water and brine, dried over Na₂SO₄, filtered and concentrated in vacuo. The residue was purified by column chromatography on SiO₂ (Hex: EtOAc=1:1˜1:2) to give to give 6-(2-fluoro-4-iodophenylamino)-N-(3-hydroxypropyl)-5-methyl-4-oxo-4,5-dihydrofuro[3,2-c]pyridine-7-carboxamide (130 mg, 9.7%) as a violet solid. ¹H-NMR (DMSO-d₆, Varian, 400 MHz) δ 1.53-1.60 (2H, m), 3.24 (q, 2H, J=6.4 Hz), 3.34 (3H, s), 3.42-3.46 (2H, m), 4.50 (1H, t), 6.60 (1H, t, J=8.8 Hz), 7.02 (1H, d, J=2.4 Hz), 7.36 (1H, dd, J=1.0 Hz), 7.63 (1H, dd, J=1.8 Hz), 7.92 (1H, d, J=2.0 Hz), 8.22 (1H, t, J=5.6 Hz), 9.93 (1H, s). m/z=486.0 [M+H]⁺.

### Biological Activity

### Materials and Preparation of Reagents:

The Kinase Glo plus assay kit was purchased from Promega. The substrate, APT, DTT, and dimethylsulfoxide were purchased from Sigma-Aldrich. The MAP2K1 (MEK1) kinase, Europium labeled Antibody, Tracer 236 and binding buffer A were purchased from Invitrogen. The Recombinant Human Epithelial Growth Factor (EGF) was purchased from R&D System. The SureFire Phospho-ERK1/2 Assay kit and the AlphaScreen General IgG (Protein A) Detection kit were both purchased from PerkinElmer.

### Generation of IC₅₀ Data

### Determination of Enzymatic Activity:

A Mek1 kinase assay (LANCE, PerkinElmer) was developed for supporting compound profiling and lead optimization. In this assay, un-phosphorylated/inactive Erk1 (Millipore) was used as the substrate for Mek1 (Millipore). Then the phosphorylated Erk1 was able to phosphorylate ULight™-MBP peptide (PerkinElmer). The phosphorylated peptide was detected by Europium-anti-phospho-MBP (PerkinElmer). In a reaction, the activity of Mek1 (0.25 nM) was measured in a buffer containing 50 μM ATP, 2 nM inactive Erk1, 50 nM ULight™-MBP peptide, and a compound for 90 min at 23° C. After quenching the reaction, 2 nM Europium-anti-phospho-MBP was added to the reaction mixture and incubated for 60 min, followed by a detection using EnVision. The IC₅₀ values were derived through a curve fitting using GraFit.

### Generation Of Cell Based IC₅₀ Data

To investigate whether a compound is able to inhibit the activity of MEK in cells, a mechanism-based assay using A375 cell line (melanoma) was developed. In this assay (AlphaScreen, PerkinElmer), inhibition of MEK was detected by reduced ERK phosphorylation. A375 cells were cultured in a tissue culture flask to 80% confluence in DMEM plus 10% fetal bovine serum. Cells were collected and plated onto 96 well culture plates at 3×10⁴ cells/well. Plates were incubated overnight at 37° C. with 5% CO₂ to allow cells to adhere. Compounds were added to the plates and incubated at 37° C. for 1 hour. After removing the medium, 100 μl of cell lysis buffer were added to each well and 4 μl of cell lysate were transferred into a 384 well white Proxiplate (PerkinElmer). The phospho-ERK levels were determined by following the standard protocol supplied with the PerkinElmer SureFire Phospho-ERK 1/2 Assay Kit®. Plates were read out by EnVision (PerkinElmer). The data were analyzed using GraphPad Prism.

### Biological Data for Select Compounds

Select compounds prepared as described above were assayed according to the biological procedures described herein. The results are given in the table below:

| | IC₅₀ (nM) | |
|---|---|---|
| Structure | Enzymatic assay | Cell-based assay (A375) |
| 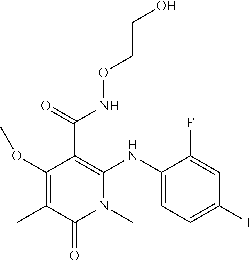 | <100 | <100 |
| 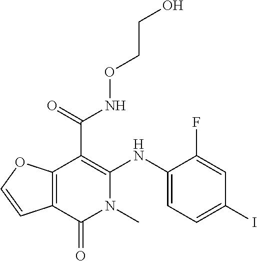 | <100 | <100 |
| 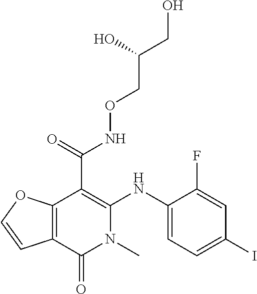 | <100 | <100 |
| 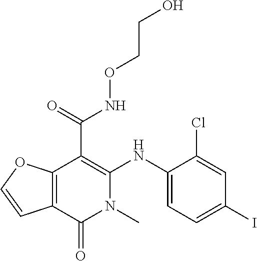 | <100 | <100 |
| 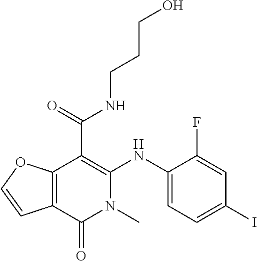 | <5000 | <100 |
| 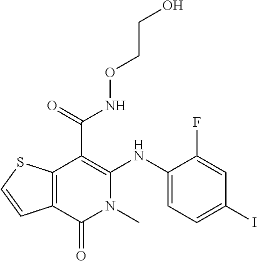 | <1000 | <100 |
| 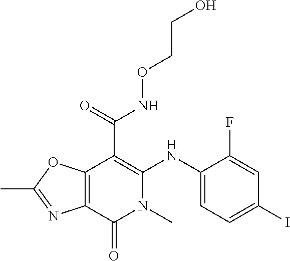 | <1000 | <100 |
| 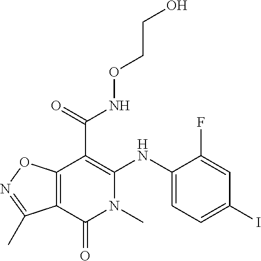 | <5000 | <100 |
| 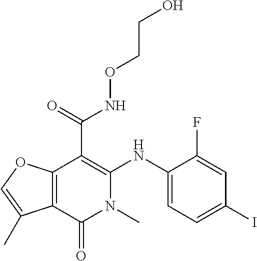 | <1000 | <100 |
| 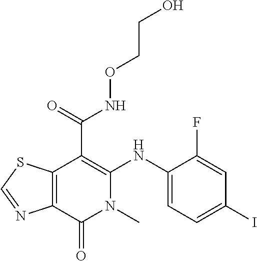 | <5000 | <5000 |

What is claimed is:

1. A compound of formula I or formula II 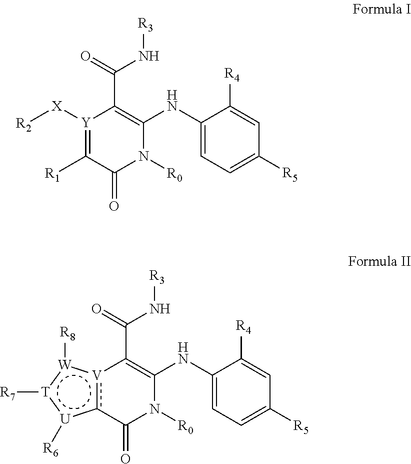
wherein
R₀ is H, C₁-C₆ alkyl, C₃-C₆ cycloalkyl, C₂-C₆ alkenyl, C₅-C₆ cycloalkenyl or C₂-C₆ alkynyl; wherein each alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, and one or two ring carbon atoms of said C₃-C₆ cycloalkyl groups are optionally replaced with, independently, O, N, or S; and
R₁ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl; or
R₁ is —CH₂X′ where X′ represents a group according to formula (III) 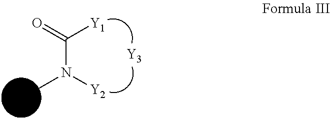
wherein
Y₁ and Y₂ may be the same or different, each represents a single bond, —CO—, —COO, —O—, —OCO—, —NRₐ or —SO₂—;
Y₃ represents a C₁₋₅ alkyl which may be substituted by one to three groups represented by Z;
Z may be the same or different and represent a C₁₋₅ alky group, halogen atom, an oxo group, —ORₐ, —COORₐ, —COOCORₐ, —CO-halogen atom, —OCORₐ, —CONRₐR_{b}, —SRₐ, —SO₂Rₐ, —NRₐR_{b}, —NRₐCOR_{b}, NRₐSO₂R_{b}, —SO₂NRₐR_{b}, a 5 or 6 membered monocyclic or 9 to 13 membered bicyclic heterocyclic group, or a 5 or 6 membered monocyclic or 9 to 13 membered bicyclic heteroaryl group which may be optionally substituted with one or more substituents selected from the group consisting of a C₁₋₅ alkyl group, —ORₐ, and NRₐR_{b}; the alkyl group may be substituted by a hydroxyl group, a C₁₋₅ alkoxy group, or an amino group; the above substituents except the oxo group and the halogen may be linked to each other to form a cycloalkyl group or a heterocyclic group which may has one or more substituents selected from the group consisting of —ORₐ, NRₐR_{b}, and a C₁₋₅ alkyl group that may be substituted with —ORₐ;
Rₐ and R_{b} may be the same or different and each represents a hydrogen atom or a C₁₋₅ alkyl group which may be substituted by one to three groups selected from the group consisting of a hydroxyl group, a C₁₋₅ alkoxy group and an amino group
The symbol “” used in formula III implies the site of bonding; and
X is O, N, S or bond;
R₂ is C₁-C₆ alkyl, C₃-C₆ cyclo alkyl, C₂-C₆ alkenyl, C₅-C₆ cycloalkenyl or C₂-C₆ alkynyl; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₂ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl when Y═C or X—R₂=nothing when Y═N; or
R₃ is selected from the group consisting of H, Me, Et, OH, OMe, EtO, HOCH₂CH₂O—, MeCH(OH)CH₂O—, HOCH₂CH(OH)CH₂O—, cyclopropyl-CH₂O—, HOCH₂CH₂O—, HOCH(CH₂CH₃)CH₂O—, HOCH₂C(CH₃)₂CH₂O—, HOCH₂C(CH₃)₂O—, HOCH(CH₃)CH₂O—, MeOCH₂CH₂O—, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heteroarycycloalkyl, heterocyclyl, and heterocyclylalkyl, where each alkyl, alkenyl, alkynyl, cycloalkyl, cycloalkylalkyl, aryl, arylalkyl, heteroaryl, heteroarylalkyl, heterocyclylalkyl, heteroarycycloalkyl, and heterocyclyl is unsubstituted or substituted with 1-3 substituents selected independently from halogen, hydroxyl, C₁-C₄ alkyl, C₁-C₄ alkoxy, cyano, trifluoromethyl, difluoromethoxy, phenyl or substituted phenyl with 1-3 substituents selected independently from halogen, hydroxyl, C₁-C₄ alkyl, C₁-C₄ alkoxy, cyano trifluoromethyl, or difluoromethoxy;
R₄ and R₅ are independently selected from hydrogen, halogen, cyano, nitro, trifluoromethyl, SR₉, OR₉, C(O)R₉, NR₁₀C(O)OR₁₂, OC(O)R₉, NR₁₀S(O)ⱼR₁₂, S(O)ⱼNR₉R₁₀, S(O)ⱼNR₁₀C(O)R₉, C(O)NR₁₀S(O)ⱼR₁₂, S(O)ⱼR₁₂, NR₁₀C(O)R₉, C(O)NR₉R₁₀, NR₁₁C(O)NR₉R₁₀, NR₁₁C(NCN)NR₉R₁₀, NR₉R₁₀ and C₁-C₁₀ alkyl, C₂-C₁₀ alkenyl, C₂-C₁₀ alkynyl, C₃-C₁₀ cycloalkyl, C₃-C₁₀ cycloalkylalkyl, S(O)ⱼ(C₁-C₆ alkyl), S(O)ⱼ(CR₁₀R₁₁)ₘ-aryl, aryl, arylalkyl, heteroaryl, heteroarylalkyl, heterocyclyl, heterocyclylalkyl, O(CR₁₀R₁₀)ₘ-aryl, NR₁₀(CR₁₀R₁₁)ₘ-aryl, O(CR₁₀R₁₁)ₘ-heteroaryl, NR₁₀(CR₁₀R₁₁)ₘ-heteroaryl, O(CR₁₀R₁₁)ₘ-heterocyclyl, NR₁₀(CR₁₀R₁₁)ₘ-heterocyclyl, and S(C₁-C₂ alkyl) optionally substituted with 1-5 fluorine atoms;
R₉ is selected from the group consisting of hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, where each alkyl, alkenyl, alkynyl, cycloalkyl, aryl, heteroaryl and heterocyclyl is unsubstituted or substituted with 1-3 substituents independently selected from the group consisting of halogen, C₁-C₄ alkyl, hydroxyl and amino;
R₁₀ is selected from hydrogen or C₁-C₆ alkyl where alkyl may be unsubstituted or substituted with 1-3 substituents independently selected from the group consisting of halogen, C₁-C₄ alkyl, hydroxyl and amino; or
R₉ and R₁₀ can be taken together with the atom to which they are attached to form a 4 to 10 membered heteroaryl or heterocyclic ring, each of which is unsubstituted or substituted with 1-3 substituents independently selected from the group consisting of halogen, C₁-C₄ alkyl, hydroxyl and amino;
R₁₁ is selected from hydrogen or C₁-C₆ alkyl where alkyl may be unsubstituted or substituted with 1-3 substituents independently selected from the group consisting of halogen, C₁-C₄ alkyl, hydroxyl and amino; or
R₁₀ and R₁₁ can be taken together with the atom to which they are attached to form a 4 to 10 membered carbocyclic, heteroaryl or heterocyclic ring, each of which is unsubstituted or substituted with 1-3 substituents independently selected from the group consisting of halogen, C₁-C₄ alkyl, hydroxyl and amino;
R₁₂ is selected from trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, where each alkyl, cycloalkyl, aryl, heteroaryl and heterocyclyl unsubstituted or substituted with 1-3 substituents independently selected from the group consisting of halogen, C₁-C₄ alkyl, hydroxyl and amino;
m is 0, 1, 2, 3, 4, or 5; and
j is 1 or 2.
T, U, V and W are each independently C, O, N or S to form a heterocycle
R₆ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when U═C and;
R₇ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when V═C and;
R₈ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when W═C and;
n is 0, 1, 2, 3 or 4
R_{c}═H, C₁-C₄, C₁-C₆ alkyl, C₃-C₆ cycloalkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; and
R_{d}═H, C₁-C₄, C₁-C₆ alkyl, C₃-C₆ cycloalkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; or
R_{c} and R_{d} taken together form a 5 or 6 membered heterocyclic group containing 1-2 heteroatoms selected independently from the group consisting of O, N or S and is optionally substituted with 1-2 substituents selectly independently form the group consisting of C₁-C₄ alkyl or C₁-C₄ alkoxy;
or a pharmaceutically acceptable salt, solvate, polymorph, ester, tautomer or prodrug thereof.

2. A compound according to claim 1 wherein
R₀ is C₁-C₆ alkyl; R₁ is H or C₁-C₆ alkyl; R₂ is C₁-C₆ alkyl; Y═C; X═O; R₃ is selected from the group consisting of H, Me, Et, OH, OMe, EtO, HOCH₂CH₂O—, MeCH(OH)CH₂O—, HOCH₂CH(OH)CH₂O—, cyclopropyl-CH₂O—, HOCH₂CH₂O—, HOCH(CH₂CH₃)CH₂O—, HOCH₂C(CH₃)₂CH₂O—, HOCH₂C(CH₃)₂O—, HOCH(CH₃)CH₂O—, MeOCH₂CH₂O—, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heteroarycycloalkyl, heterocyclyl, and heterocyclylalkyl, where each alkyl, alkenyl, alkynyl, cycloalkyl, cycloalkylalkyl, aryl, arylalkyl, heteroaryl, heteroarylalkyl, heterocyclylalkyl, heteroarycycloalkyl, and heterocyclyl is unsubstituted or substituted with 1-3 substituents selected independently from halogen, hydroxyl, C₁-C₄ alkyl, C₁-C₄ alkoxy, cyano, trifluoromethyl, difluoromethoxy, phenyl or substituted phenyl with 1-3 substituents selected independently from halogen, hydroxyl, C₁-C₄ alkyl, C₁-C₄ alkoxy, cyano trifluoromethyl, or difluoromethoxy;
T is C or N, U is C or N; V is C or N; W is C or O; R₆ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when U═C; R₇ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when V═C; R₈ is H, C₁-C₄ alkoxy, C₁-C₆ alkyl, C₃-C₆ cycloalkyl, C₂-C₆ alkenyl, C₅-C₆ cycloalkenyl C₂-C₆ alkynyl, or halogen; wherein each alkoxy, alkyl, cycloalkyl, alkenyl, cycloalkenyl or alkynyl group is optionally substituted with 1-3 substituents selected independently from the group consisting of halogen, hydroxy, C₁-C₄ alky, C₁-C₄ alkoxy, cyano, cyanomethyl, trifluoromethyl, difluoromethoxy and phenyl, or R₁ is 5 or 6-atom heterocyclic group, which group may be saturated, unsaturated, or aromatic, containing 1-5 heteroatoms selected independently from the group consisting of O, N, and S, which heterocyclic group is optionally substituted with 1-3 substituents selected independently from the group consisting of halogen, hydroxy, C₁-C₄ alky, C₁-C₄ alkoxy, (CH₂)ₙNR_{c}R_{d}, cyano, cyanomethyl, trifluoromethyl, difluoromethoxy and phenyl when W═C and;
n=0, 1, 2, 3 or 4
R_{c}═H, C₁-C₄, C₁-C₆ alkyl, C₃-C₆ cycloalkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; and
R_{d}═H, C₁-C₄, C₁-C₆ alkyl, C₃-C₆ cycloalkyl, C₂-C₆ alkenyl, C₅-C₆ cycloalkenyl C₂-C₆ alkynyl, or halogen; wherein each alkyl, cycloalkyl, alkenyl, cycloalkenyl or alkynyl group is optionally substituted with 1-3 substituents selected independently from the group consisting of C₁-C₄ alky, C₁-C₄ alkoxy, trifluoromethyl, difluoromethoxy and phenyl; or
R_{c} and R_{d} taken together form a 5 or 6 membered heterocyclic group containing 1-2 heteroatoms selected independently from the group consisting of O, N or S and is optionally substituted with 1-2 substituents selectly independently form the group consisting of C₁-C₄ alkyl or C₁-C₄ alkoxy; and
R₄ and R₅ are independently selected form H and halogen;
or a pharmaceutically acceptable salt, solvate, polymorph, ester, tautomer or prodrug thereof.

3. The compound according to claim 1 having the formula 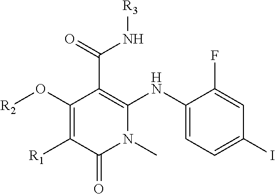
wherein
R₁, R₂ and R₃ are defined as claim 1;
or a pharmaceutically acceptable salt, solvate, polymorph, ester, tautomer or prodrug thereof.

4. The compound according to claim 1 having the formula 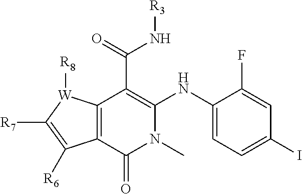
wherein
R₃, R₆, R₇ and R₈ are defined as claim 1;
or a pharmaceutically acceptable salt, solvate, polymorph, ester, tautomer or prodrug thereof.

5. A pharmaceutical composition comprising a pharmaceutically effective amount of a compound of any of claims 1 to 4 or a pharmaceutically acceptable salt, solvate, polymorphpolymorph, ester, tautomer or prodrug thereof, and a pharmaceutically acceptable carrier.

6. Use of a compound of any of claims 1 to 4 or a pharmaceutically acceptable salt, solvate, polymorph, ester, tautomer or prodrug thereof in the preparation of a pharmaceutical composition for inhibiting MEK enzyme.

7. Use of a compound of any of claims 1 to 4 or a pharmaceutically acceptable salt, solvate, polymorph, ester, tautomer or prodrug thereof in the preparation of a pharmaceutical composition for the treatment or prophylaxis of a MEK mediated disorder or disease.

8. Use of a compound of any of claims 1 to 4 or a pharmaceutically acceptable salt, solvate, polymorph, ester, tautomer or prodrug thereof in the preparation of a pharmaceutical composition for the treatment or prophylaxis of proliferative disorders.

9. Use of claim 8, wherein the proliferative disorders are selected from the group consisting of inflammatory diseases and cancers.

10. A method for inhibiting a MEK enzyme comprising the step of contacting the MEK enzyme with an amount sufficient to inhibit said enzyme of a compound of any of claims 1 to 4 or a pharmaceutically acceptable salt, solvate, polymorph, ester, tautomer or prodrug thereof.

11. A method for the treatment or prophylaxis of a MEK mediated disorder or disease comprising administering to an individual in need thereof an effective amount of a composition comprising a compound of any of claims 1 to 4 or a pharmaceutically acceptable salt, solvate, polymorph, ester, tautomer or pro-drug thereof.

12. The method of claim 11, wherein the disorder or disease is proliferative disorders.

13. The method of claim 12, wherein the proliferative disorders are selected from the group consisting of inflammatory diseases and cancers.
